(19) United States
(12) Patent Application Publication
(10) Pub. No.: US 2008/0096799 A1
(43) Pub. Date: Apr. 24, 2008

(54) COMPOUNDS FOR AND METHODS OF TREATING INSULIN RESISTANCE AND INFLAMMATION

(75) Inventor: John Nestor, Encinitas, CA (US)

Correspondence Address: KIRTON AND MCCONKIE, 60 EAST SOUTH TEMPLE,, SUITE 1800, SALT LAKE CITY, UT, 84111, US

(73) Assignee: FORBES MEDI-TECH (RESEARCH), INC., Seattle, WA (US)

(21) Appl. No.: 11/851,935

(22) Filed: Sep. 7, 2007

Related U.S. Application Data
(60) Provisional application No. 60/824,897, filed on Sep. 7, 2006.

(51) Int. Cl. A61K 38/28; A61P 3/10; C07D 239/02; A61K 31/4965

(52) U.S. Cl. 514/3; 544/335; 514/256

(57) ABSTRACT

Novel compounds, compositions comprising compounds, and methods for preparing and using compounds are described herein. Methods of treating or ameliorating various conditions, including insulin resistance, pancreatic beta cell apoptosis, obesity, pro-thrombotic conditions, myocardial infarction, hypertension, dyslipidemia, manifestations of Syndrome X, congestive heart failure, inflammatory disease of the cardiovascular system, atherosclerosis, restenosis, sepsis, type 1 diabetes, liver damage, COPD, emphysema and cachexia, by administering compounds described herein. Compounds presented herein may be used to modulate serine palmitoyl transferase activity.

### CROSS-REFERENCE TO RELATED APPLICATIONS

This application claims the benefit of U.S. Provisional Application No. 60/824,897, filed Sep. 7, 2006, which application is incorporated herein by reference.

### BACKGROUND

All publications mentioned herein are cited for the purpose of familiarizing the reader with the background of the invention. Nothing herein is to be construed as an admission that these references are prior art in relation to the inventions described herein.

Although Type 2 Diabetes (i.e., T2D, diabetes mellitus, non-insulin dependent diabetes mellitus, adult onset diabetes) is frequently thought of as a disease caused by high blood sugar, modern thinking has regarded blood glucose levels as mainly a symptom of an underlying disease related to dysregulated fat metabolism. Thus high fatty acid levels lead to a range of lipotoxicities: insulin resistance, pancreatic beta cell apoptosis, and a disorder termed “metabolic syndrome.” In addition, and as discussed below, there is increasing recognition that these lipotoxicities are part of and encompass a broader range of inflammatory syndromes (Unger R. H. Annu Rev Med 53: 319-36 (2002)). Insulin resistance can be detected by the following indications: as an increased level of blood insulin, increased blood levels of glucose in response to oral glucose tolerance test (OGTT), decreased levels of phosphorylated protein kinase B (AKT) in response to insulin administration, and the like. Insulin resistance may be caused by decreased sensitivity of the insulin receptor-related signaling system in cells and/or by loss of beta cells in the pancreas through apoptosis. There is also evidence that insulin resistance can be characterized as having an underlying inflammatory component (Grundy, S. M., et al. Circulation 109: 433-8 (2004)).

Sedentary lifestyle and obesity have contributed to the increased occurrence of T2D. Therapeutic intervention has been aimed at people with impaired glucose tolerance (IGT). IGT is defined as hyperglycaemia (with glucose values intermediate between normal and diabetes) following a glucose load, and affects at least 200 million people worldwide. People afflicted with IGT possess a higher future risk than the general population for developing diabetes. Approximately 40% of people with IGT progress to diabetes in 5-10 years, but some revert to normal or remain IGT.

Moreover, people with IGT also have a heightened risk of developing cardiovascular disease, such as hypertension, dyslipidaemia and central obesity. Thus, the diagnosis of IGT, particularly in apparently healthy and ambulatory individuals, has important prognostic implications. For a more detailed review, see Zimmet P, et al., Nature, 414:783-7 (2001), the disclosure of which is incorporated herein by reference.

Recently, impaired fasting glucose (IFG) is introduced as another category of abnormal glucose metabolism. IGF is defined on the basis of fasting glucose concentration and, like IGT, it is also associated with risk of cardiovascular disease and future diabetes.

T2D may be caused by a variety of factors. Additionally, the disease also manifests heterogeneous symptoms. Previously, T2D was regarded as a relatively distinct disease entity, but current understanding has revealed that T2D (and its associated hyperglycaemia or dysglycaemia) is often a manifestation of a much broader underlying disorder, which includes the metabolic syndrome. This syndrome is sometimes referred to as Syndrome X, and is a cluster of cardiovascular disease risk factors that, in addition to glucose intolerance, includes hyperinsulinaemia, atherogenic dyslipidaemia, hypertension, visceral obesity, hypercoagulability, and microalbuminuria.

Recent understanding of the factors leading to T2D has influenced contemporary therapy for the disease. More aggressive approaches to treating hyperglycaemia as well as other risk factors such as hypertension, dyslipidaemia and central obesity in type 2 diabetics have been pursued. In addition, more simplistic and comprehensive screening of at-risk individuals has been advocated by health organizations, such as the American Diabetes Association.

Ceramide has been reported as showing activity in some of the factors relating to T2D, such as insulin resistance and beta cell apoptosis. For example, Schmitz-Peiffer et al. report that feeding cells with palmitic acid or ceramide leads to insulin resistance (Schmitz-Peiffer C. et al., J. Biol. Chem., 274: 24202-10 (1999)). Increased levels of palmitic acid in cells leads directly to increased levels of ceramide through an increase in levels of Palmitoyl-CoA which feeds into the de novo ceramide synthesis pathway. Studies suggest that de novo ceramide synthesis of ceramide is an important factor, since inhibition of ceramide synthase with fuminosin blocks beta cell apoptosis (Shimabukuro M., et al., Proc. Natl. Acad. Sci. USA, 95: 2498-2502 (1998)). Similarly, it has been recognized that the enzyme involved in the rate limiting step for the de novo pathway for ceramide synthase, serine palmitoyl transferase (SPT), may be a viable target for blockade of beta cell apoptosis. For example, Shimabukuro et al. report that inhibition of SPT with cycloserine has a partial beta cell protective effect (≈50% activity) in the diabetic Zucker fatty rat model (Shimabukuro et al., J. Biol. Chem., 273: 32487-90 (1998), the disclosure of which is incorporated herein by reference).

As mentioned above, atherogenic dyslipidemia is part of the metabolic syndrome and atherosclerosis is a major human disease. It is now recognized that atherosclerosis has an important inflammatory component. In an intriguing series of studies with the SPT inhibitor myriocin the observation was made that a dramatic reduction in atherosclerotic plaque was observed (Park, et al. Circulation 110: 3456-71 (2004); Hojjati, et al. J. Biol. Chem. 280: 10284-9 (2005); Park, T S, Panek, R. L., Rekhter, M. D., Mueller, S. B., Rosebury, W. S., Robertson, A. W, Hanselman, J. C. (2006). Modulation of lipoprotein metabolism by inhibition of sphingomyelin synthesis in ApoE knockout mice. Atherosclerosis, epub ahead of print (2006). While the authors are tempted to ascribe the observed plaque reduction to inhibition of SPT, these studies with myriocin do not convincingly demonstrate this result, as acknowledged by the authors. This is due to the other major biological activity of myriocin, inhibition of lymphocyte chemotaxis. This latter effect is the cause of the known potent immunosuppressive activity of myriocin and offers a confounding possibility to the research reported above. This activity is caused by the phosphorylation of myriocin in vivo to generate a structure that mimics the structure and activity of sphingosine-1-phosphate (S1P). This structure binds to Edg receptors to inhibit release of lymphocytes from the spleen. These activities are mimicked by the immunosuppressive FTY720 and much of the mechanism has been clarified using FTY720 and its analogs (Rosen, H. and Liao, J. Curr. Opin. Chem. Biol. 7: 461-8 (2003)).

As noted above, use of myriocin or compounds substantially structurally similar to myriocin with immunosuppressive activity may not be an attractive approach to an antiatherosclerosis therapeutic and there is a need for alternative compounds and methods. The compounds of the invention, with their clean SPT inhibitory or modulative activity and minimal action at Edg receptors or little cross-reactivity with Edg receptors, offer clear therapeutic advantages over myriocin and related compounds.

An important class of treatments for acute coronary disease is that referred to as Percutaneous Coronary Intervention (PCI). PCI means a group of existing and developing therapies that are used to treat acute coronary disease: percutaneous transluminal coronary angioplasty, rotational atherectomy, directional atherectomy, etraction atherectomy, laser angioplasty, implantation of intracoronary stents and other catheter devices for treating vessel narrowing fall within this classification (Smith S. C., et al. ACC/AHA Guidelines for Percutaneous Coronary Intervention (Revision of the 1993 PTCA Guidelines)—Executive Summary. Circulation 103: 3019-3041 (2001)). Restenosis after stenting is a critical problem and is thought to have an important inflammatory component (Gaspardone A and Versaci, F. Coronary stenting and inflammation. Am. J. Cardiol. 96(12A): 65L-70L (2005)). Recent research indicates that agents such as the HMG-CoA reductase inhibitors (Statins) can have anti-inflammatory activities and that this aspect can have important beneficial effects when given in conjunction with PCI (Gaspardone, A, et al. Effect of atorvastatin (80 mg) initiated at the time of coronary artery stent implantation on C-reactive protein and six-month clinical events. Am. J. Cardiol. 90:786-9 (2002)). Additional support for this anti-inflammatory mechanism of action is provided by the demonstration of beneficial effects from the local administration of dexamethasone from drug eluting stents on clinical outcome (Radke, P. W., et al. Dexamethasone and restenosis after coronary stent implantation. Curr. Pharm. Des. 10: 3449-55 (2004)). There are many factors to consider in novel stent design including materials, coatings and active agent (Wittaker, D. R. and Fillinger, M. F. The engineering of endovascular stent technology: a review. Vasc. Endovascular Surg. 40: 85-94 (2006); Yang, C. and Burt, H. M. Drug-eluting stents: factors governing local pharmacokinetics. Adv. Drug Deliv. Rev. 58: 402-11 (2006); Burt, H. M. and Hunter, W. L. Drug-eluting stents: a multidisciplinary success story. Adv. Drug Deliv. Rev. 58: 350-7 (2006)). While the most prominent drug eluting stents make use of cytostatic (Burke, S. E., et al. Zotarolimus (ABT-578) eluting stents. Adv. Drug Deliv. Rev. 58:437-46 (2006)) or immunosuppressive agents, there is a clear involvement of inflammatory processes in the restenosis problem and a continuing need for improvement over existing stents (Desmet, W. Delayed neointimal healing after drug-eluting stent implantation: seeing is believing. Eur. Heart J. epub ahead of print (2006)).

A well known pro-inflammatory signal, Tumor Necrosis Factor alpha (TNF), has been shown to raise ceramide levels in cells in culture (Sawada, M, et al. Cell Death Differ. 11, 997-1008 (2004); Meyer, S G, et al. Biochim Biophys Acta. 1643(1-3), 1-4 (2003)). TNF administration reduces PPAR-gamma levels in adipocytes and this has been shown to implicate ceramide (Kajita, K, et al. Diabetes. Res. Clin. Pract. 66 Suppl 1, S79-83 (2004)). TNF also induces apoptosis in liver cells and has been implicated in injury due to viral hepatitis, alcoholism, ischemia, and fulminant hepatic failure (Ding, W X and Yin, X M, J. Cell. Mol. Med. 8, 445-54 (2004); Kanzler S., et al. Semin Cancer Biol. 10(3):173-84 (2000)). Similarly, TNF and IL-6 are implicated in cachexia, another syndrome with strong evidence of an inflammatory component, implicating ceramide as an effector. It is known that atherosclerosis has an inflammatory component. Induction of oxidative stress by amyloid involves induction of a cascade that increases ceramide levels in neuronal cells (Ayasolla K., et al. Free Radic. Biol. Med. 37(3):325-38 (2004)). Thus altered ceramide levels may be causative in dementias such as Alzheimer's disease and HIV dementia and modulation of these levels with an SPT inhibitor is conceived as having promise as a treatment (Cutler R G, et al. (2004). Proc Natl. Acad. Sci. 101, 2070-5.). TNF is known to be involved in sepsis and insulin has protective effects (Esmon, C T. Crosstalk between inflammation and thrombosis. Maturitas. 47, 305-14 (2004)). De novo ceramide levels possibly serve as a central effector mechanism in the inflammatory processes central to many diseases and conditions. However, the potential for modulators of SPT to be used as therapeutic agents for diseases and conditions related to ceramide's involvement, as an effector in inflammatory processes, has not previously been shown.

Pulmonary emphysema, which with chronic bronchitis comprises chronic obstructive pulmonary disease (COPD), is an important lung disease characterized by difficulty in breathing effectively due to airspace enlargement caused by tissue destruction. Alveolar apoptosis is recognized as playing an important role in the development of emphysema. In a mouse model of emphysema driven by inhibition of VEGF receptor function one sees alveolar cell apoptosis, oxidative stress and airspace destruction. These symptoms were shown to be blocked by treatment with fumonisin B1 or myriocin, inhibitors of de novo ceramide synthesis (Petrache I., et al., (2005). Ceramide upregulation causes pulmonary cell apoptosis and emphysema-like disease in mice. Nat. Med. 11: 491-8). Thus blockade of de novo ceramide synthesis by compounds of the invention will comprise a novel treatment for emphysema and COPD without the side effects expected for treatment with fumonisin B1 or myriocin.

Elevated levels of fatty acids can induce a syndrome that mimics the pathology of cardiomyopathy (i.e., heart failure). The pathogenesis of this lethal condition is poorly understood, but appears to be related to lipotoxicities. Studies indicate that lipid overload in cardiac myocytes may well be an underlying cause for cardiomyopathy. In addition, recent studies have identified low levels of myocyte apoptosis (80-250 myocytes per 10⁵ nuclei) in failing human hearts. It remains unclear, however, whether this cell death is a coincidental finding, a protective process, or a causal component in disease pathogenesis (See, e.g., Wencker D. et al., J. Clin. Invest., 111:1497-1504 (2003), the disclosure of which is incorporated herein by reference). Increases in fatty acid levels in cells directly lead to elevated rates of de novo ceramide synthesis. TNF has been implicated in CHF, and thereby ceramide, an associated effector for TNF signaling, is implicated through an independent direction (McTiernan, C F, et al. Curr Cardiol Rep. 2(3), 189-97 (2000)). However, the utility of de novo ceramide synthesis modulators, as agents to block progression of and allow healing of heart muscles in cardiomyopathy, has not been demonstrated.

Cachexia is a progressive wasting syndrome with loss of skeletal muscle mass (Frost R A and Lang C H.; Curr Opin Clin Nutrit Metab Care. 2005; 255-263) and adipose tissue. This syndrome is found in response to infection, inflammation, cancer (Tisdale M J; Langenbecks Arch Surg. 2004; 389: 299-305) or some chronic diseases like rheumatoid arthritis (Rall L C and Roubenoff R Rheumatol 2004: 43, 1219-23). Release of various cytokines has been implicated in this syndrome and both TNF and IL-6 are recognized as central players. Thus cachexia can be looked at as a chronic inflammatory state. Ceramide is a well-known central effector of TNF signaling. In addition, ceramide is known to modulate the expression of IL-6 (Shinoda J, Kozawa O, Tokuda H, Uematsu, T.; Cell Signal. 1999; 11: 435-41; Coroneos, E; Wang, Y; Panuska, J R; Templeton, D J; Kester, M.; Biochem J 1996; 316: 13-7). Existing data lead us to believe that de novo ceramide synthesis is playing a central role as a signal for this inflammatory state as well. We therefore believe that inhibition of TNF and/or IL-6 signaling through ceramide will provide a clinical benefit to patients with this wasting syndrome.

Rosenberg and others have shown that isolation of pancreatic islets for transplantation, e.g., for use in the treatment of diabetes, is made difficult by the low yields that result from isolation and that these low yields are due in significant measure to beta cell apoptosis. Structural and functional changes resulting from islet isolation lead to islet cell death (Rosenberg L, Wang R, Paraskevas S, Maysinger D. Surgery. 126: 393-8 (1999); Cell loss in isolated human islets occurs by apoptosis. Paraskevas S, Maysinger D, Wang R, Duguid T P, Rosenberg L; Pancreas. 20(3): 270-6 (2000); Challenges facing islet transplantation for the treatment of type 1 diabetes mellitus. Rother K I, Harlan D M J Clin Invest. 114, 877-83 (2004)).

Beattie, et al have reported that various treatments (e.g. trehalose, removal of Arg from culture medium, and the like) may improve the yield of transplantable islets but substantial cell death remains (Beattie G M, Leibowitz G, Lopez A D, Levine F, Hayek A. Cell Transplant. 9:431-8 (2000)). Treatment of cells and tissues by caspase inhibitors leads to a partial block of apoptosis in response to various metabolic insults, but apoptosis may be driven by many mechanisms, and caspase inhibition may have useful or marginal effects depending on the specific instance being studied (Biotechnol Bioeng., 81:329-40 (2003)). Study of caspase inhibitors for limiting death in mammalian cell culture (Sauerwald T M, Oyler G A, Betenbaugh M J. Biotechnol. Bioeng. 81: 329-40 (2003)).

Studies of inhibition of de novo synthesis of ceramide have shown that such inhibition appears to have anti-apoptotic effects in a number of important situations. Beta cell apoptosis in response to treatment with free palmitic acid and/or in combination with high levels of glucose can be blocked by treatment with fumonisin B1 (inhibitor of ceramide synthase), for example (Maedler, K. Diabetes; 52:726-33 (2003). It is thus possible that the inhibition or de novo ceramide synthesis can be applied to prevention of apoptotic events. However, treatment with agents that inhibit ceramide synthase have been shown to result in toxic effects, as seen with ingestion of fumonisin B1 (Bennett J W and Klich M. Clin Microbiol Rev. 16, 497-516 (2003)). Inhibition of SPT provides an alternate method for preventing apoptosis of pancreatic beta cells, however, modulators of SPT have not been shown to prevent the loss of pancreatic beta cells in culture prior to transplant.

Thus, modulators of de novo ceramide synthesis could provide important new therapeutic agents for a range of human and veterinary diseases that entail an inflammatory component making use of ceramide as an effector agent. However, interference with the de novo ceramide synthesis pathway at several points (e.g., as with Fumonisin B1) is known to lead to toxicities. Inhibition at the level of Serine Palmitoyl Transferase, however, leads to the build up of innocuous cellular components serine and Palmitoyl CoA.

Known inhibitors of SPT include cycloserine, D-serine, myriocin, sphingofungin B, viridiofungin A, and lipoxamycin. A number of these natural products, such as myriocin, have been shown to have unacceptable toxicities. Furthermore, these ceramides impart only partially protective activity. In addition, some SPT inhibitors, such as cycloserine, show weak inhibition and exhibit low specificity. Structural studies suggest that natural ceramides mimic the active site bound form of the starting materials or products (Hanada K. et al., Biochem Biophys Acta, 1632: 16-30 (2003)).

Myriocin is known to be a powerful immunosuppressive molecule as well as an inhibitor of SPT. A number of analogs have been designed based on its structure. Structures that have the immunosuppressive activity of myriocin, such as those related to compound FTY720, illustrated below, do not inhibit SPT. Additionally, the carboxylic derivative of FTY720, shown below as compound 2, did not exhibit activity against SPT, as demonstrated in an immunosuppressive assay for FTY720-like activity (Kiuchi M. et al., J. Med. Chem., 43: 2946-61 (2000)) and was suggested to be inactive due to extremely low solubility if not lack of binding affinity, per se.

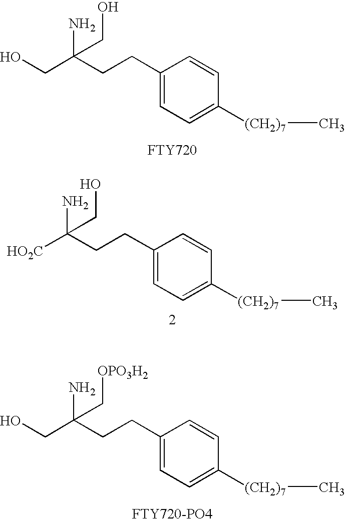

Work with FTY720 has demonstrated that it undergoes phosphorylation by sphingosine kinase and that the resulting phosphorylated species (FTY720-PO4) is the active molecule in vivo (Mandala S. et al., Science 296: 346-9 (2002); Brinkmann V. et al. J. Biol. Chem. 277: 21453-7 (2002); Rosen H and Liao, J. Curr. Opin. Chem. Biol. 7: 461-8 (2003)). Thus the source of the immunomodulatory activity inherent in the structure of myriocin is the hydroxymethyl function on the head group which can be phosphorylated to yield an S1P like structure.

Modulation of SPT presents an attractive means to attenuate insulin resistance and prevent loss of pancreatic beta cells. Inhibitors of SPT, in particular, may offer new therapeutics for the treatment of T2D. These agents could be beneficial for the protection of tissue for transplantation such as in islet transplantation and liver transplantation. As outlined above, such inhibitors could also have beneficial uses in the treatment of cardiomyopathy, sepsis, cachexia atherosclerosis, liver damage, reperfusion injury, Alzheimer's Disease, Type 1 diabetes, in which apoptosis plays a role, as well as other inflammatory diseases. Bioavailable agents that are highly potent and selective inhibitors of SPT, especially with respect to lack of S1P activity, were heretofore not available. Nontoxic, bioavailable, potent and selective modulators of SPT could prove to be important new agents for the treatment of the diseases and conditions as disclosed herein and other diseases and conditions involving apoptosis and in which TNF is known, to those of skill in the art, to play a role. The generation of such compounds and their usefulness for treating these indications has not been previously shown.

### SUMMARY OF THE INVENTION

Presented herein are novel compounds and methods of use. In a preferred embodiment, compounds provided herein exhibit activity on the enzyme, serine palmitoyl transferase (SPT) and lack the potential to be phosphorylated on the 2 position side chain, which could lead to S1P-like activity.

Presented herein are novel compounds, and pharmaceutically acceptable salts thereof, corresponding to Formula (I): 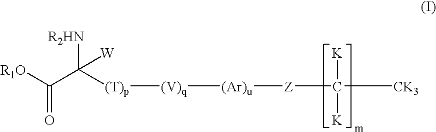
wherein:
R₁ is H, or optionally substituted lower alkyl, aryl, aralkyl, or alkyloxyalkyl;
R₂ is H, protecting group, or —C(═O)—CHRₐ—NHR_{b};
   Rₐ is selected from the group consisting of alkyl, aralkyl, aryl, and optionally substituted alkyl with hydroxyl, halo, alkenyl, alkynl, ether, thiol, methylthio, borate, boronate, phospho, phosphono, phosphine, heterocyclic, enone, imine, aldehyde, ester, thioacid, hydroxylamine, amino group, and combinations thereof;
   R_{b} is H or amino protecting group;
W is R₅ or selected from the group consisting of —CH₃, —CH₂CH₃, —CH₂X, —CHX₂, —CX₃, —CH₂CH₂X,
   —CH₂CHX₂, —CH₂CX₃, —CH₂SO₂NH—R₃, —CH₂CH₂SO₂NH—R₃, —CH₂NHSO₂—R₃, —CH₂CH₂NHSO₂NH—R₃,
   —CH₂SO₂—R₃, —CH₂CH₂SO₂—R₃, —C(O)OH, —CH₂C(O)OH, —CH₂C(O)NH—R₃, —CH₂CH₂C(O)NH—R₃, —CH₂NHC(O)—R₃,
   —CH₂CH₂NHC(O)—R₃, —CH₂NHC(O)NH—R₃, —CH₂CH₂NHC(O)NH—R₃, —CH₂O—R₄, —CH₂CH₂O—R₄,
   —CH₂NO₂, —CH₂CH₂NO₂, —CH₂CN, —CH₂CH₂CN, —CH₂OCX₃, —CH₂CH₂OCX₃, and —CH₂C(O)CX₃;
   each X is independently a halogen;
   R₃ is H, or optionally substituted lower alkyl, aryl, aralkyl, or alkyloxyalkyl;
   R₄ is optionally substituted lower alkyl, aryl, aralkyl, or alkyloxyalkyl;
   R₅ is optionally substituted lower alkyl, aryl, aralkyl, or alkyloxyalkyl, with the proviso that R₅ is not
      —CH₂OR₂;
each V and Z is independently (CR_{c}R_{d})ₖ, CR_{c}═CR_{d}, O, NR_{c}, S, Ar, CR_{c}R_{d}Ar, OAr, NR₄Ar, SAr, or ArAr;
   each R_{c}, and R_{d} is independently H, lower alkyl, OH, or O-lower alkyl;
   or R_{c}, and R_{d} together form a ═O, ═N—OH, ═N—O-lower alkyl, or ═N—O—CH₂CH₂—O—CH₃;
   Rₑ is H, lower alkyl, or —CH₂CH₂—O—CH₃;
   k is 1 to 7;
q is 1 to 13;
each K is independently —H, —OH, —X, or —CH₃;
each T is independently (CR_{f}R_{g});
   each R_{f} is independently H, lower alkyl, or O-lower alkyl;
   each R_{g} is independently OH, or O-lower alkyl;
   or R_{f} and R_{g}, together form a ═O, ═N—OH, ═N—O-lower alkyl, or ═N—O—CH₂CH₂—O—CH₃;
p is 1 to 5;
each Ar is an optionally substituted aryl or heteroaryl;
u is 0, 1, or 2; and
m is 4 to 12.

Compounds provided herein may be employed in the treatment of a variety of human diseases or conditions. In a preferred embodiment, compounds are used to treat diseases such as T2D, insulin resistance, pancreatic beta cell apoptosis, or obesity. In another preferred embodiment, compounds are used to treat pro-thrombotic conditions, congestive heart failure, myocardial infarction, hypertension, atherogenic dyslipidemia, or other symptoms of Metabolic Syndrome (i.e., Syndrome X). In yet another preferred embodiment, compounds are used to treat inflammatory diseases, such as inflammatory diseases of the cardiovascular system, sepsis and cachexia. Exemplary inflammatory diseases of the cardiovascular system include atherosclerosis. In yet another preferred embodiment, these compounds are used to prevent liver damage from viral, alcohol related, reperfusion injuries as outlined above. In yet another preferred embodiment, these compounds are used to protect and enhance the yield for transplantation of pancreatic liver cells and or livers, either alone or in combination with the currently approved cocktails and/or caspase inhibitors. In yet another preferred embodiment, these compounds are used to treat inflammatory lung diseases such as emphysema and COPD.

Also provided are compositions comprising compounds presented herein, in combination with a therapeutically effective amount of another active agent. Exemplary agents include insulin, insulin analogs, incretin, incretin analogs, glucagon-like peptide, glucagon-like peptide analogs, exendin, exendin analogs, PACAP and VIP analogs, sulfonylureas, biguanides, α-glucosidase inhibitors, Acetyl-CoA Carboxylase inhibitors, caspase inhibitors, delta 3 unsaturated fatty acids, polyunsaturated fatty acids and PPAR ligands. Accordingly, embodiments of methods for treating various diseases include co-administering compounds presented herein and a therapeutically effective amount of another active agent, or administration of combination compositions provided herein.

### DETAILED DESCRIPTION

As described above, the compounds of the invention inhibit SPT, the first committed step of an enzymatic pathway known to have a broad pro-inflammatory role as an effector of TNFα signaling. Therefore, modulation of this pathway has great importance for the treatment of a number of inflammatory diseases, for example—the Metabolic Syndrome (Syndrome X) and its components (atherosclerosis, insulin resistance, prothrombotic state, hypertension), diabetes (beta cell apoptosis; in vitro and in vivo), congestive heart failure, sepsis, cachexia, liver damage (inflammatory or viral), restenosis, drug eluting stents, and the like.

Furthermore, the agents of the invention can be used advantageously in combination with other known therapeutics for these diseases for even greater beneficial effect. This includes use in conjunction with 1. insulin or insulin analogs (human, hog, beef, lispro, aspart, glargine, detemir), 2. oral hypoglycemic agents such as the sulfonylureas and the agents having similar effect (Glipizide, Gliclazide, Glibenclamide, Glimepiride, Repaglinide, Nateglinide and the generic chemical forms thereof), 3. Biguanides (metformin, buformin, phenformin, and the like), 4. alphaglucosidase inhibitors (Acarbose, miglitol, and the like), 4. caspase inhibitors (VX-765, IDN-6556, and the like), 5. PPAR ligands (pioglitazone, rosiglitazone, and the like, including ligands of all PPAR receptor classes), 6. Incretin/GlP1 analogs (exenatide, Liraglutide, ZP-10A/AVE-010, Albugon, BIM-51077 and the like), 7. PACAP or VIP analogs (Ro 25-1555, Bay 55-9837, and the like), and 8. Acetyl-CoA inhibitors. These examples are meant to be illustrative and not limit the scope of the combinations of therapeutics contemplated by the invention.

As mentioned above, a major biological activity of myriocin is immunosuppression caused by inhibition of lymphocyte chemotaxis. This activity is caused by the phosphorylation of myriocin in vivo to generate a structure that mimics the structure and activity of sphingosine-1-phosphate (S1P). This structure binds to Edg receptors to interfere with the release of lymphocytes from the spleen. This immunosuppressive activity of myriocin and its analogs may be an undesirable attribute for some of the uses described herein. Therefore the compounds of the invention are differentiated from myriocin and analogs by being designed to inhibit SPT activity, but to have strongly diminished immunosuppressive activity.

There are a number of assays that can be used to determine whether a molecule has potent immunosuppressive activity through the mode of action used by myriocin and FTY720 (Chiba, K., et al. Role of Sphingosine 1-Phosphate Receptor Type 1 in Lymphocyte Egress from Secondary Lymphoid Tissues and Thymus. Cell. Molec. Immunol. 3: 11-19 (2006)). A simple in vivo assay uses the quantitation of lymphocytes 24 hr after treatment of normal rats and makes use of flow cytometry to determine amounts of T-cells and B-cells in the peripheral blood (Kiuchi, M., et al. Synthesis and Immunosuppressive Activity of 2-Substituted 2-Aminopropane-1,3-diols and 2-Aminoethanols. J. Med. Chem. 43: 2946-61 (2000)). Kiuchi, et al (2000) also report the use of a rat skin allograft model and popliteal lymph node gain assays. FTY720 may be used as a positive control and less than 10% or preferably less than 1% of the activity of FTY720, is indicative of weak immunosuppressive activity, which is desirable for the compounds of the invention.

As used in the specification, “a” or “an” means one or more. As used in the claim(s), when used in conjunction with the word “comprising,” the words “a” or “an” mean one or more. As used herein, “another” means at least a second or more.

Reference now will be made in detail to various embodiments and particular applications of the invention. While the invention will be described in conjunction with the various embodiments and applications, it will be understood that such embodiments and applications are not intended to limit the invention. On the contrary, the invention is intended to cover alternatives, modifications and equivalents that may be included within the spirit and scope of the invention. Where a particular structure is disclosed herein that has potential stereoisomer, the structure incorporates, each individual stereoisomer above, both stereoisomers together and any mixture of any ratio of the two, as appropriate. In addition, throughout this disclosure various patents, patent applications, websites and publications are referenced, and unless otherwise indicated, each is incorporated by reference in its entirety for all purposes. All publications mentioned herein are cited for the purpose of describing and disclosing reagents, methodologies and concepts with the present invention. Nothing herein is to be construed as an admission that these references are prior art in relation to the inventions described herein.

### I. COMPOUNDS

Presented herein are novel compounds, and pharmaceutically acceptable salts thereof, corresponding to Formula (I): 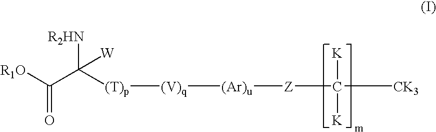
wherein:
R₁ is H, or optionally substituted lower alkyl, aryl, aralkyl, or alkyloxyalkyl;
R₂ is H, protecting group, or —C(═O)—CHRₐ—NHR_{b};
   Rₐ is selected from the group consisting of alkyl, aralkyl, aryl, and optionally substituted alkyl with hydroxyl, halo, alkenyl, alkynl, ether, thiol, methylthio, borate, boronate, phospho, phosphono, phosphine, heterocyclic, enone, imine, aldehyde, ester, thioacid, hydroxylamine, amino group, and combinations thereof;
   R_{b} is H or amino protecting group;
W is R₅ or selected from the group consisting of —CH₃, —CH₂CH₃, —CH₂X, —CHX₂, —CX₃, —CH₂CH₂X,
   —CH₂CHX₂, —CH₂CX₃, —CH₂SO₂NH—R₃, —CH₂CH₂SO₂NH—R₃, —CH₂NHSO₂—R₃, —CH₂CH₂NHSO₂NH—R₃,
   —CH₂SO₂—R₃, —CH₂CH₂SO₂—R₃, —C(O)OH, —CH₂C(O)OH, —CH₂C(O)NH—R₃, —CH₂CH₂C(O)NH—R₃, —CH₂NHC(O)—R₃,
   —CH₂CH₂NHC(O)—R₃, —CH₂NHC(O)NH—R₃, —CH₂CH₂NHC(O)NH—R₃, —CH₂O—R₄, —CH₂CH₂O—R₄,
   —CH₂NO₂, —CH₂CH₂NO₂, —CH₂CN, —CH₂CH₂CN, —CH₂OCX₃, —CH₂CH₂OCX₃, and —CH₂C(O)CX₃;
   each X is independently a halogen;
   R₃ is H, or optionally substituted lower alkyl, aryl, aralkyl, or alkyloxyalkyl;
   R₄ is optionally substituted lower alkyl, aryl, aralkyl, or alkyloxyalkyl;
   R₅ is optionally substituted lower alkyl, aryl, aralkyl, or alkyloxyalkyl, with the proviso that R₅ is not
      —CH₂OR₂;
each V and Z is independently (CR_{c}R_{d})ₖ, CR_{c}═CR_{d}, O, NRₑ, S, Ar, CR_{c}R_{d}Ar, OAr, N Ar, SAr, or ArAr;
   each R_{c}, and R_{d} is independently H, lower alkyl, OH, or O-lower alkyl;
   or R_{c}, and R_{d} together form a ═O, ═N—OH, ═N—O-lower alkyl, or ═N—O—CH₂CH₂—O—CH₃;
   Rₑ is H, lower alkyl, or —CH₂CH₂—O—CH₃;
   k is 1 to 7;
q is 1 to 13;
each K is independently —H, —OH, —X, or —CH₃;
each T is independently (CR_{f}R_{g});
   each R_{f} is independently H, lower alkyl, or O-lower alkyl;
   each R_{g} is independently OH, or O-lower alkyl;
   or R_{f} and R_{g}, together form a ═O, ═N—OH, ═N—O-lower alkyl, or ═N—O—CH₂CH₂—O—CH₃;
p is 1 to 5;
each Ar is an optionally substituted aryl or heteroaryl;
u is 0, 1, or 2; and
m is 4 to 12.

Preferred compounds of Formula (I) include those where R₁ is lower alkyl, such as methyl, ethyl, isopropyl, and the like. Additionally preferred embodiments include those compounds where R₁ is alkyloxyalkyl, such as CH₃—O—CH₂—CH₂—, HO—CH₂—CH₂—O—, HO—(CH₂—CH₂—O—)ⱼ—, hydroxyethyl alcohol, hydroxypropyl alcohol, hydroxyethyloxyethyl alcohol, and polyethylene glycol or derivatives thereof. Other preferred compounds of Formula (I) include those where X is halogen, such as fluorine. Additional preferred compounds of Formula (I) include those where Z is NR₄, O, or S. Another preferred embodiment includes compounds of Formula (I) where Ar is an optionally substituted heteroaryl. Another preferred embodiment includes compounds of Formula (I) where Ar is an optionally substituted fused ring system, such as a 5-5, 5-6, or 6-6 ring system. Also additional preferred embodiments include those compounds where W is selected from the group consisting of —CH₂SO₂NH₂, —CH₂C(O)NH₂, —CH₂SO₂NHCH₃, —CH₂CN, —CH₂CF₃, —CH₂OCH₃, —CH₂OCF₃, —CH₃, —CH₂F, —CF₂H, —CH₂CCl₃, —CH₂C(O)NHCH₃, CH₂SO₂CH₃,

—CH₂NHC(O)H, —CH₂NHC(O)NH₂, and —CH₂C(O)CF₃.

In an embodiment, compounds of Formula (I) correspond to Formula (II): 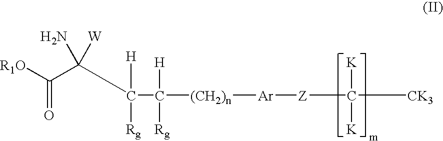
wherein n is 0 to 7.

In an embodiment, compounds of Formulas (I) and (II) correspond to Formula (IIA): 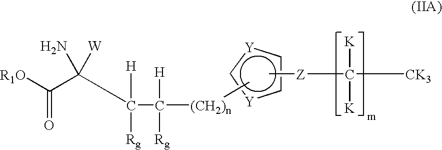
wherein each Y is independently C, CH, O, S, N, or NH.

In another embodiment, compounds of Formulas (I) and (II) correspond to Formula (IIB): 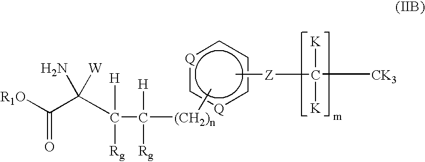
wherein each Q is independently C, CH, N, or NH.

In yet another embodiment, compounds of Formulas (I) and (II) correspond to Formula (IIC): 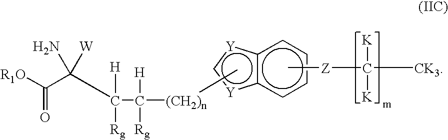
wherein each Y is independently C, CH, O, S, N, or NH.

In another embodiment, compounds of Formulas (I) and (II) correspond to Formula (IID):

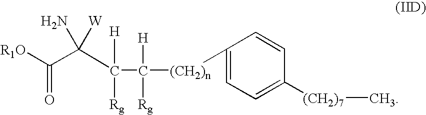

In another embodiment, compounds of Formulas (I) and (II) correspond to Formula (IIE):

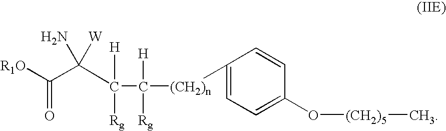

In another embodiment, compounds of Formulas (I) and (II) correspond to Formula (IIF):

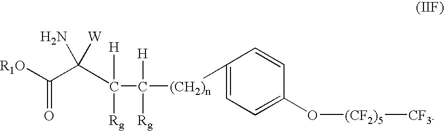

In an additional embodiment, compounds of Formula (I) correspond to Formula (III): 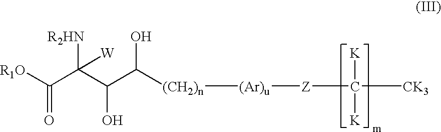
wherein n is 0 to 7.

In another embodiment, compounds of Formula (I) correspond to Formula (IIIA): 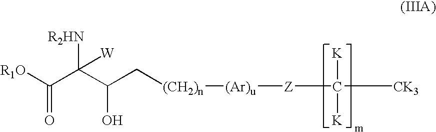
wherein n is 0 to 7.

In another embodiment, compounds of Formula (I) correspond to Formula (IIIB): 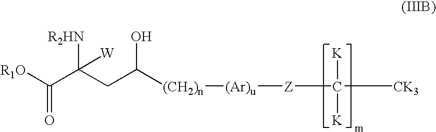
wherein n is 0 to 7.

In another embodiment, compounds of Formula (I) correspond to Formula (IIIC): 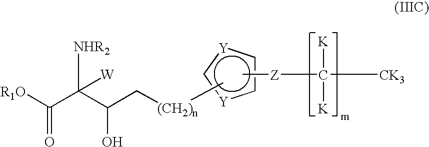
wherein each Y is independently C, CH, O, S, N, or NH; and n is 0 to 7.

In another embodiment, compounds of Formula (I) correspond to Formula (IIID): 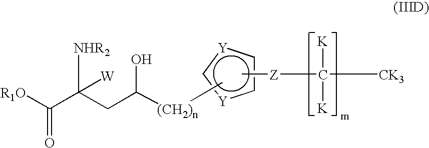
wherein each Y is independently C, CH, O, S, N, or NH; and
n is 0 to 7.

In another embodiment, compounds of Formula (I) correspond to Formula (IIIE): 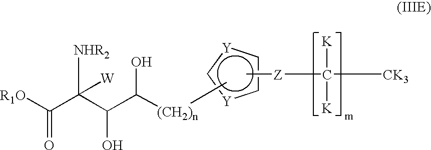
wherein each Y is independently C, CH, O, S, N, or NH; and
n is 0 to 7.

In another embodiment, compounds of Formula (I) correspond to Formula (IIIF): 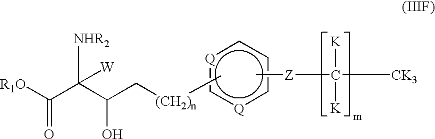
wherein each Q is independently C, CH, N, or NH; and
n is 0 to 7.

In another embodiment, compounds of Formula (I) correspond to Formula (IIIG): 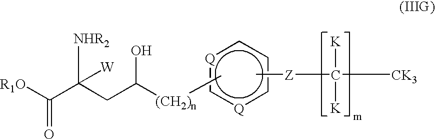
wherein each Q is independently C, CH, N, or NH; and
n is 0 to 7.

In another embodiment, compounds of Formula (I) correspond to Formula (IIIH): 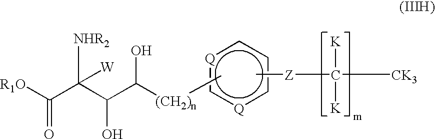
wherein each Q is Independently C, CH, N, or NH; and
n is 0 to 7.

In another embodiment, compounds of Formula (I) correspond to Formula (IIIJ): 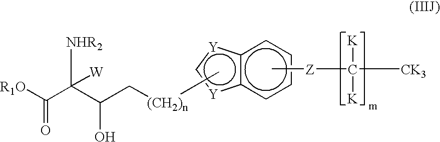
wherein each Y is independently C, CH, O, S, N, or NH; and
n is 0 to 7.

In another embodiment, compounds of Formula (I) correspond to Formula (IIIK): 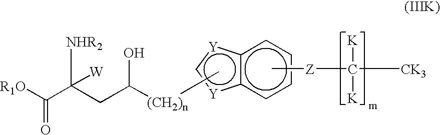
wherein each Y is independently C, CH, O, S, N, or NH; and
n is 0 to 7.

In another embodiment, compounds of Formula (I) correspond to Formula (IIIL): 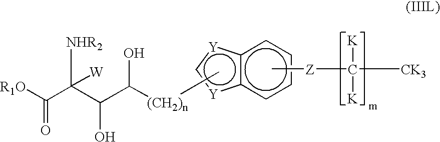
wherein each Y is independently C, CH, O, S, N, or NH; and
n is 0 to 7.

In another embodiment, compounds of Formula (I) correspond to Formula (IIIM): 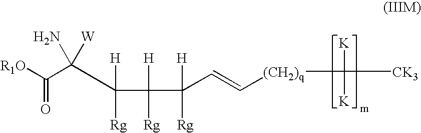
wherein q plus m is less than 12.

In another embodiment, compounds of Formula (I) correspond to Formula (IIIN):

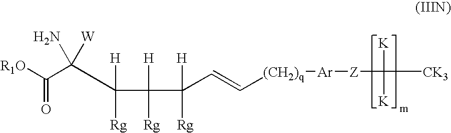

In yet another embodiment, prodrug forms of compounds of Formula (I) are presented. Prodrug forms of compounds are optimal for oral administration, and typically correspond to the ester of the acid active species. Active species of the prodrugs can be used to prepare active drug compounds.

In an embodiment, prodrug compounds correspond to Formula (IIIO): 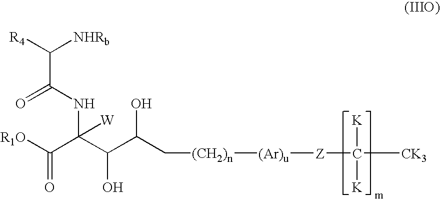
wherein Rₐ is the side chain of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, pyrolysine and selenocysteine; and n is 0 to 7.

Representative prodrug compounds corresponding to Formula (IIIO) include compounds corresponding to Formula (IIIP):

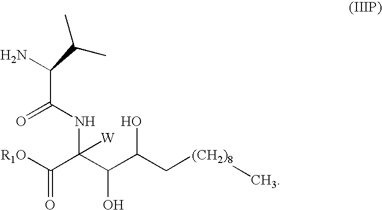

In another embodiment, prodrug compounds correspond to Formula (IIIQ): 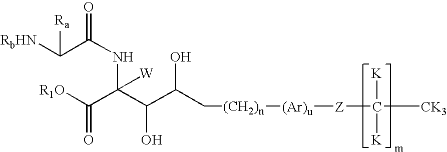
wherein Rₐ is the side chain of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, pyrolysine and selenocysteine; and n is 0 to 7.

Representative prodrug compounds corresponding to Formula (IIIP) include compounds corresponding to Formula (IIIR):

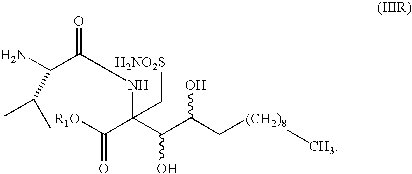

As mentioned above, myriocin's known potent immunosuppressive activity is caused by the phosphorylation of myriocin in vivo to generate a structure that mimics the structure and activity of sphingosine-1-phosphate (S1P). This structure binds to Edg receptors to inhibit release of lymphocytes from the spleen. These activities are mimicked by the immunosuppressive FTY720 and much of the mechanism has been clarified using FTY720 and its analogs (Rosen, H. and Liao, J. Curr. Opin. Chem. Biol. 7: 461-8 (2003)). Compounds of this invention can prevent the above-described phosphorylation in vivo for causing immunosuppressive activity. Thus, compounds of this invention do not cause strong immunosuppressive activity.

Structures that are capable of inhibiting SPT but of not being phosphorylated to yield an immunosuppressive moiety would have advantages over myriocin and other phosphorylatable analogs due to increased selectivity and decreased potential for side effects. The compounds of the invention contain polar side chain substitutions off of position 2 in place of the OH functional group found on myriocin. Thus these substitutions are designed to be bioisosteric (Lima, L. M. and Barreiro, E. J. (2005). Curr. Med. Chem. 12: 23-49; Patani, G. A., LaVoie, E. J. (1996) Chem Rev 96: 3147; Chen, X, Wang, W. (2003) Ann. Rep. Med. Chem. 38: 333; the disclosures of all of which are incorporated herein by reference) with the hydroxymethyl group of myriocin. Thus, in certain embodiments, W does not comprise hydroxymethyl. In certain embodiments, W is not —CH₂OR₂.

In another embodiment, compounds of Formula (I) correspond to Formula (IVA): 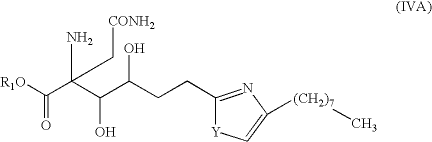
wherein Y is C, CH, O, S, N, or NH.

In another embodiment, compounds of Formula (I) correspond to Formula (IVB):

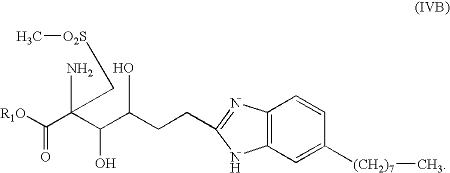

In another embodiment, compounds of Formula (I) correspond to Formula (IVC):

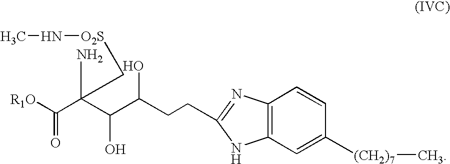

In another embodiment, compounds of Formula (I) correspond to Formula (IVD):

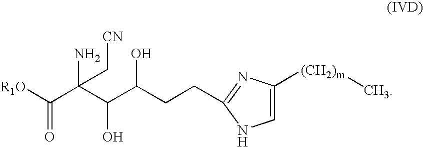

In another embodiment, compounds of Formula (I) correspond to Formula (IVE):

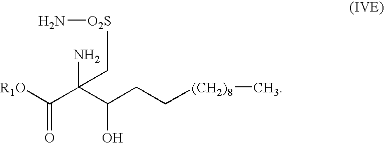

In another embodiment, compounds of Formula (I) correspond to Formula (IVF):

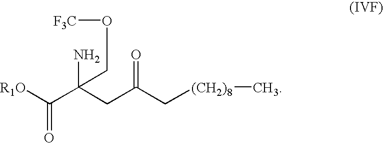

In another embodiment, compounds of Formula (I) correspond to Formula (IVG):

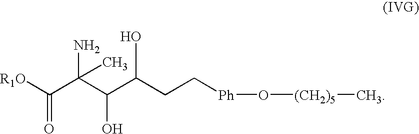

In another embodiment, compounds of Formula (I) correspond to Formula (IVH):

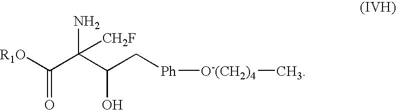

In another embodiment, compounds of Formula (I) correspond to Formula (IVJ):

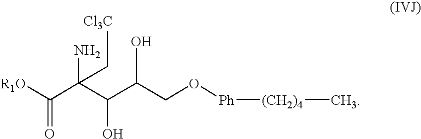

In another embodiment, compounds of Formula (I) correspond to Formula (IVK):

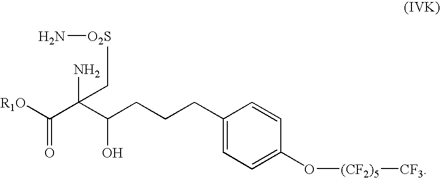

In another embodiment, compounds of Formula (I) correspond to Formula (IVL):

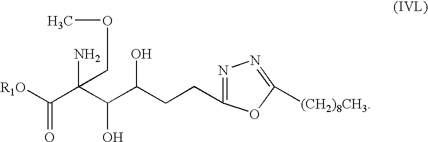

In another embodiment, compounds of Formula (I) correspond to Formula (IVM):

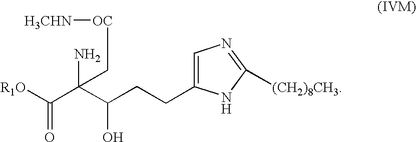

In another embodiment, compounds of Formula (I) correspond to Formula (IVN):

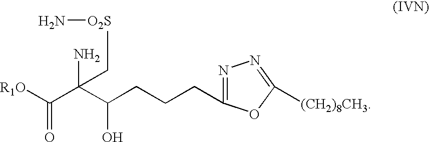

In another embodiment, compounds of Formula (I) correspond to Formula (IVP):

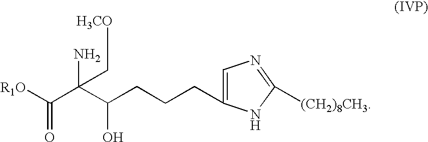

In another embodiment, compounds of Formula (I) correspond to Formula (IVQ):

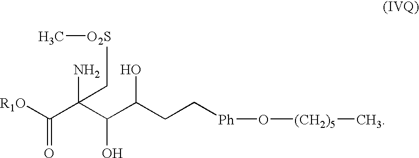

In another embodiment, compounds of Formula (I) correspond to Formula (IVR):

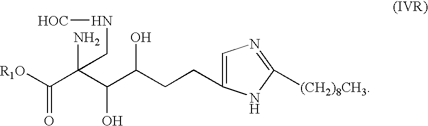

In another embodiment, compounds of Formula (I) correspond to Formula (IVS):

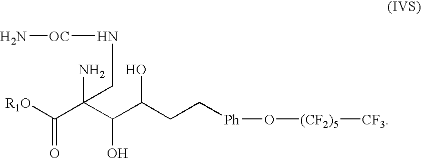

In another embodiment, compounds of Formula (I) correspond to Formula (IVT):

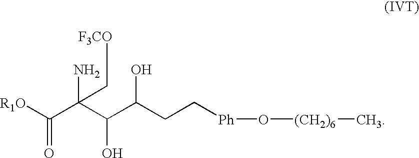

In another embodiment, compounds of Formula (I) correspond to Formula (IVU):

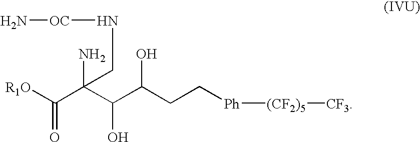

Exemplary compounds provided herein are listed below in Table 1.

TABLE 1
Representative Compounds

| | |
|---|---|
| 3 | 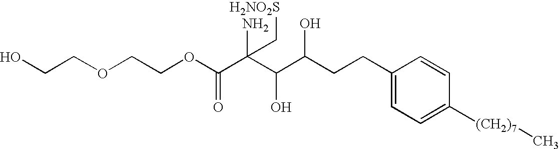 |
| 4 | 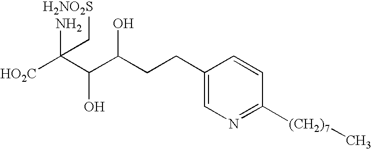 |
| 5 | 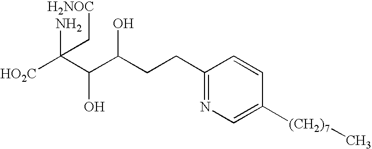 |
| 6 | 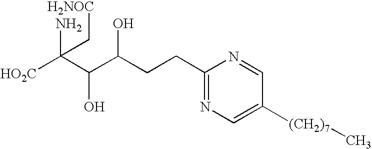 |
| 7 | 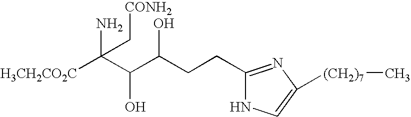 |
| 8 | 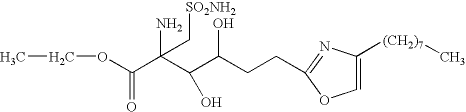 |
| 9 | 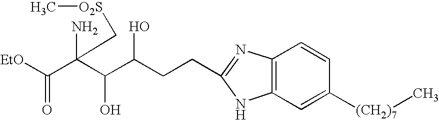 |
| 10 | 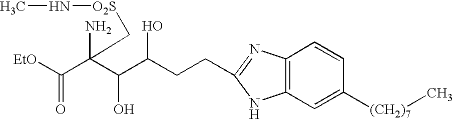 |
| 11 | 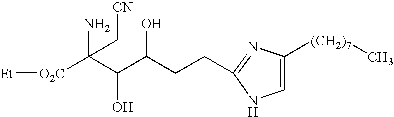 |
| 12 | 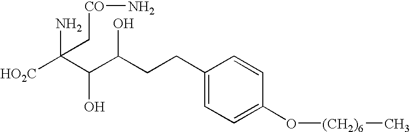 |
| 13 | 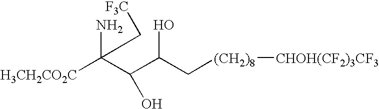 |
| 14 | 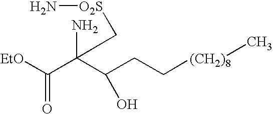 |
| 15 | 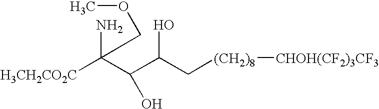 |
| 16 | 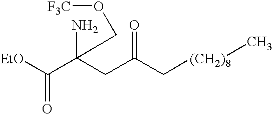 |
| 17 | 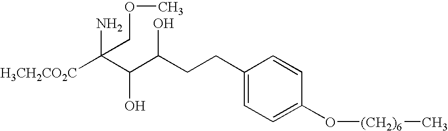 |
| 18 | 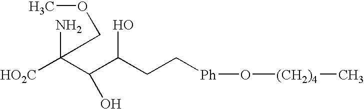 |
| 19 | 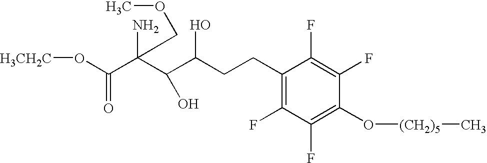 |
| 20 | 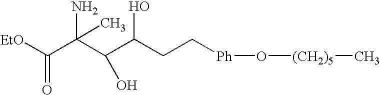 |
| 21 | 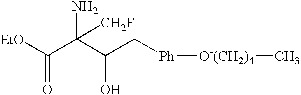 |
| 22 | 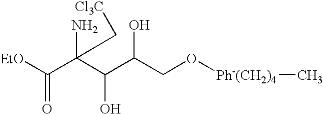 |
| 23 | 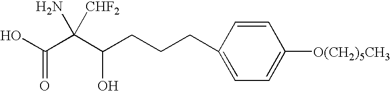 |
| 24 | 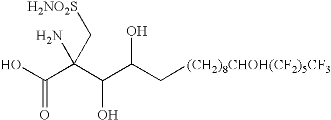 |
| 25 | 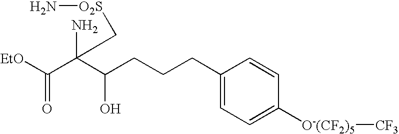 |
| 26 | 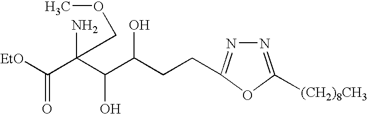 |
| 27 | 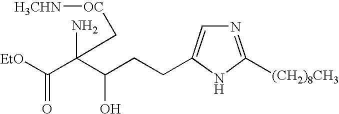 |
| 28 | 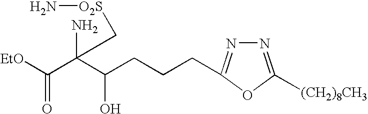 |
| 29 | 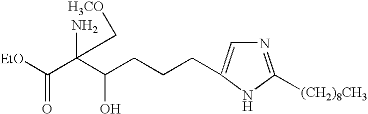 |
| 30 | 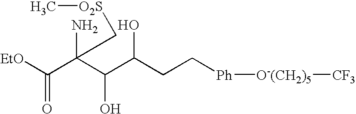 |
| 31 | 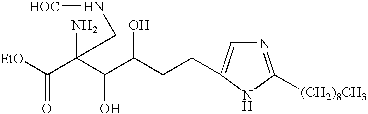 |
| 32 | 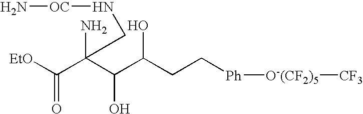 |
| 33 | 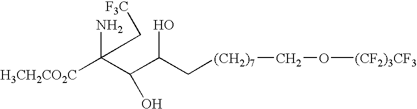 |
| 34 | 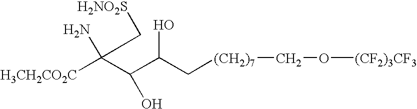 |
| 35 | 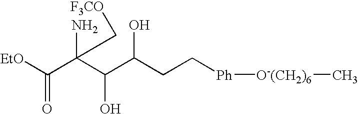 |
| 36 | 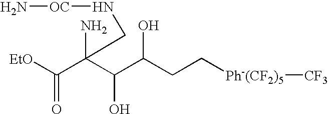 |
| 37 | 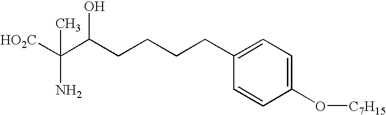 |
| 38 | 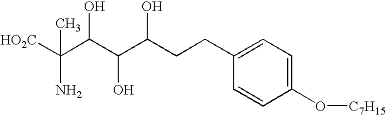 |
| 39 | 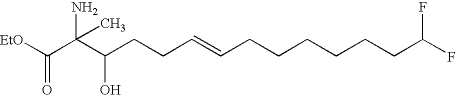 |
| 40 | 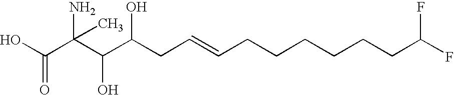 |
| 41 | 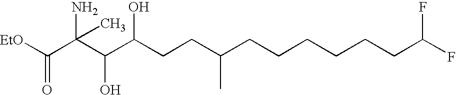 |
| 42 | 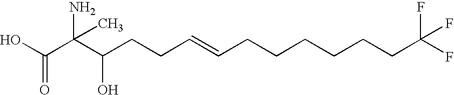 |
| 43 | 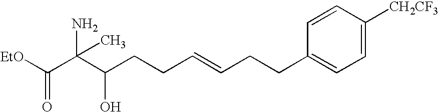 |
| 44 | 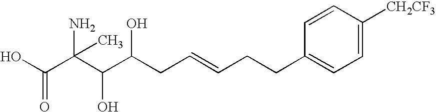 |
| 45 | 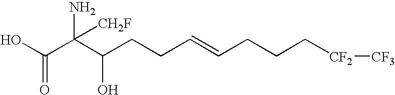 |
| 46 | 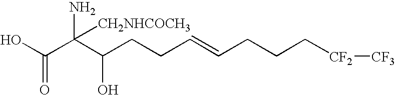 |
| 47 | 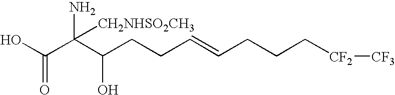 |
| 48 | 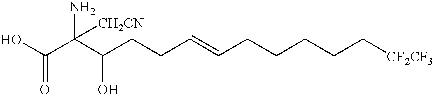 |
| 49 | 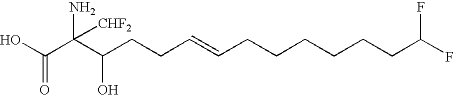 |
| 50 | 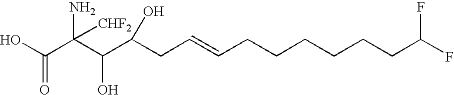 |
| 51 | 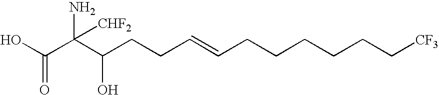 |
| 52 | 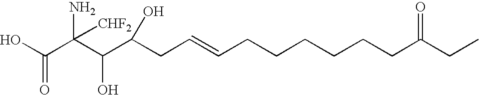 |
| 53 | 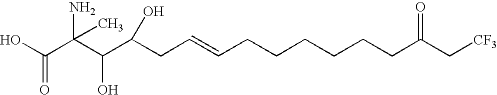 |
| 54 | 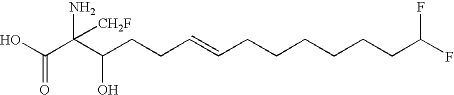 |
| 55 | 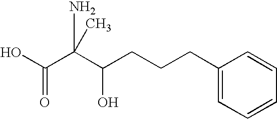 |
| 56 | 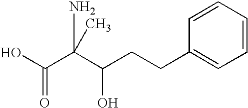 |
| 57 | 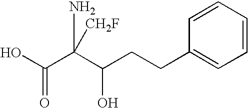 |
| 58 | 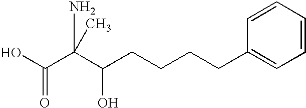 |
| 59 | 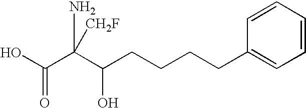 |
| 60 | 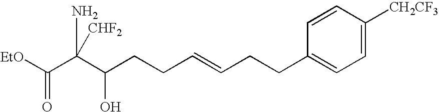 |
| 61 | 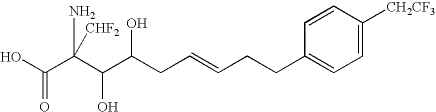 |
| 62 | 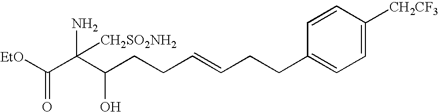 |
| 63 | 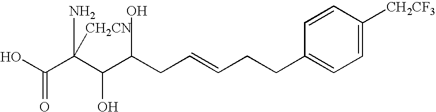 |
| 64 | 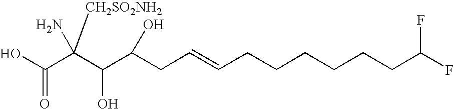 |

### II. DEFINITIONS

Compounds presented herein embrace isotopically-labelled compounds, which are identical to those recited in Formula (I), but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as 2H, ³H, ¹³C, ¹⁴C, ¹⁵N, ¹⁸O, ¹⁷O, ³⁵S, ¹⁸F, ³⁶Cl, respectively. Compounds presented herein, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. ³H and ¹⁴C isotopes are preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds herein and prodrugs thereof can generally be prepared by carrying out the procedures disclosed in the Schemes and/or in the Examples below, by substituting a readily available isotopically labelled reagent for a non-isotopically labelled reagent.

Some of the compounds herein have asymmetric carbon atoms and can therefore exist as enantiomers or diastereomers. Diasteromeric mixtures can be separated into their individual diastereomers on the basis of their physical chemical differences by methods known, for example, by chromatography and/or fractional crystallization. Enantiomers can be separated by converting the enantiomeric mixture into a diasteromeric mixture by reaction with an appropriate optically active compound (e.g., alcohol), separating the diastereomers and converting (e.g., hydrolyzing) the individual diastereomers to the corresponding pure enantiomers. Enantiomers can also be synthesized using asymmetric reagents, for example to prepare the alpha alkyl amino acid head group of myriocin and its analogs (e.g. Seebach, D. et al. (1987). Helv. Chim. Acta. 70. 1194-1216; Hale, J J, et al. (2004). Bio-org. Med. Chem. Lett., 12, 4803-7; Kobayashi, S., et al. (1998). J. Am. Chem. Soc. 120, 908-19). Alternatively, chiral synthesis of enantiomeric centers using chiral synthons from natural products is a facile approach to such syntheses, for example the synthesis of myriocin from d-mannose (Oishi, T., et al. (2001). Chemical Commun. 1932-3; and references to myriocin synthesis therein) and of myriocin analogs from isolated, natural myriocin (Chen, J K, et al. (1999). Chem. Biol. 6, 221-35; Fujita, T, et al. (1996) J. Med. Chem. 39, 4451-59). In addition, use of enzymes (free or supported) to preferentially modify one of the enantiomeric centers and thus allow separation or interconversion of enantiomers is well-known to the art (for example Wang, Y.-F., et al. (1988). J. Am. Chem. Soc. 110, 7200-5) and has great usefulness in production of pharmaceuticals. All such isomers, including diastereomers, enantiomers, and mixtures thereof are considered as part of this invention.

Those skilled in the art will recognize that some of the compounds herein can exist in several tautomeric forms. All such tautomeric forms are considered as part of this invention. Also, for example all enol-keto forms of any compounds herein are included in this invention.

Some of the compounds of this invention are acidic and may form a salt with a pharmaceutically acceptable cation. Some of the compounds of this invention can be basic and accordingly, may form a salt with a pharmaceutically acceptable anion. All such salts, including di-salts are within the scope of this invention and they can be prepared by conventional methods. For example, salts can be prepared simply by contacting the acidic and basic entities, in either an aqueous, non-aqueous or partially aqueous medium. The salts are recovered either by filtration, by precipitation with a non-solvent followed by filtration, by evaporation of the solvent, or, in the case of aqueous solutions, by lyophilization, as appropriate.

In addition, compounds herein embrace metabolites, hydrates, or solvates thereof and all of which are within the scope of the invention.

The term “substituted” refers to substitution on any carbon or heteroatom with any chemically feasible substituent. Representative substitutions include halogen substitution or substitution with any heteroatom containing group, e.g., alkoxy, phosphoryl, sulfhydryl, etc.

The term “alkyl” refers to straight chain, branched, or cyclic hydrocarbons. Exemplary of such alkyl groups (assuming the designated length encompasses the particular example) are methyl, ethyl, propyl, isopropyl, butyl, sec-butyl, tertiary butyl, pentyl, isopentyl, neopentyl, tertiary pentyl, 1-methylbutyl, 2-methylbutyl, 3-methylbutyl, hexyl, isohexyl, heptyl and octyl. The term “lower alkyl” refers to alkyl as defined above comprising C₁-C₂₀. Substituted alkyl refers to alkyl groups which are substituted as defined above and are exemplified by haloalkyl, e.g., CF₃, CHF₂, CH₂F, etc.

The term “aryl” refers to any aromatic group comprising C₃-C₂₀. Aryl groups also embrace fused ring systems, such as 5-5, 5-6, and 6-6 ring systems. Representative aryl groups include phenyl, biphenyl, anthracyl, norbornyl, and the like. Aryl groups may be substituted according to the definition provided above.

The term “heteroaryl” refers to any aryl group comprising at least one heteroatom within the aromatic ring. Heteroaryl groups also embrace fused ring systems, such as 5-5, 5-6, and 6-6 ring systems. Representative heteroaryl groups include imidazole, thiazole, oxazole, phenyl, pyridinyl, pyrimidyl, imidazolyl, benzimidazolyl, thiazolyl, oxazolyl, isoxazolyl, benzthiazolyl, or benzoxazolyl. Heteroaryl groups may be substituted according to the definition provided above.

The term “aralkyl” or “arylalkyl” refers to an aryl group comprising an alkyl group as defined above. Aralkyl or arylalkyl groups may be appended from the aryl or the alkyl moiety.

The term “alkoxy” refers to alkyl groups bonded through an oxygen. Exemplary alkoxy groups (assuming the designated length encompasses the particular example) are methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy and octoxy. Alkoxy may be substituted according to the definition provided above.

The term “alkoxyalkyl” refers to an alkoxy group comprising an alkyl group as defined above. Alkoxyalkyl groups may be substituted according to the definition provided above.

The term “halogen” refers to chloro, bromo, iodo, or fluoro.

The term “modulator” means a molecule that interacts with a target either directly or indirectly. The interactions include, but are not limited to, agonist, antagonist, and the like.

The term “agonist” means a molecule such as a compound, a drug, an enzyme activator or a hormone that enhances the activity of another molecule or the activity of a receptor site.

The term “antagonist” means a molecule such as a compound, a drug, an enzyme inhibitor, or a hormone, that diminishes or prevents the action of another molecule or the activity of a receptor site.

The terms “effective amount” or “therapeutically effective amount” refer to a sufficient amount of the agent to provide the desired biological result. That result can be reduction and/or alleviation of the signs, symptoms, or causes of a disease, or any other desired alteration of a biological system. For example, an “effective amount” for therapeutic use is the amount of the composition comprising a compound as disclosed herein required to provide a clinically significant decrease in a disease. An appropriate “effective” amount in any individual case may be determined by one of ordinary skill in the art using routine experimentation.

As used herein, the terms “treat” or “treatment” are used interchangeably and are meant to indicate a postponement of development of diseases and/or a reduction in the severity of such symptoms that will or are expected to develop. The terms further include ameliorating existing disease symptoms, preventing additional symptoms, and ameliorating or preventing the underlying metabolic causes of symptoms.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

### III. PREPARATION OF COMPOUNDS

Compounds provided herein can be prepared by synthetic methods well known to those skilled in the art. Much work has been devoted to the synthesis of α,α-disubstituted α-amino acids and these methods or extensions are applicable to certain compounds of the invention which will be apparent to those of skill in the art. Exemplary references discussing representative preparative methods which may be employed for construction of the amino acid head group region of the compounds include: Ohfune, Y. and Shinada, T. Eur. J. Org. Chem. 2005: 5127-43; Najera, T., et al. Eur. J. Org. Chem. 2000: 2809-20; Hayes, C. J., et al. J. Org. Chem. 71: 2661-5 (2006); Cativiela, C., and Diaz-de Villegas, M. D. Tetrahedron: Asymmetry 9: 3517-3599 (1998); Lee, K. Y, et al. Tet. Lett. 43: 9361-9363 (2002); Hatakeyama, S, et al. J. Org. Chem. 62: 2775-9 (1997); Trost, B. M. J. Org. Chem. 69: 5813-37 (2004); Lane, J. W. and Halcomb, R. L. Org. Lett. 5: 4017-20 (2003); the disclosures of all of which are incorporated herein by reference. Exemplary references discussing representative preparative methods which may be employed include: Kiuchi et al., J. Med. Chem., 43: 2946-61 (2000); Seidel G. et al., J. Org. Chem., 69: 3950-52 (2004); Clemens J. J. et al., Bioorg. Med. Chem. Lett., 14: 4903-6 (2004); Durand, P. et al., Synthesis, 505:6 (2000); Hale et al., Bioorg. Med. Chem. Lett., 14, 3351-5 (2004); Seebach, D., et al, Helv. Chim. Acta. 70, 1194-1216 (1987); Oishi, T, et al. Chem. Commun. 1932-3 (2001); Wang, Y.-F., et al. J. Am. Chem. Soc. 110, 7200-5 (1988); Pipik, B, et al. Synth. Commun. 34, 1863-70 (2004); the disclosures of all of which are incorporated herein by reference. Approaches to the natural products which inhibit SPT have been reviewed by Byun, H-S et al. (Synthesis 2447-74 (2006)) and these references are relevant to approaches to formation of the polar head group of the compounds of the invention. A particularly relevant study is that of Hinterding K., et al (Tetrahedron Lett. 43: 8095-7) who produce alpha alkyl analogs of FTY720 using the Schollkopf bislactim method. Similarly, studies by Kobayashi (1998a and b) are very relevant to the procedures listed below; disclosures of all of which references above are incorporated herein by reference. Additional exemplary references which may be employed relate to multicomponent assembly of amino acid like compounds: Sugiyama, S., et al. Chem. Pharm. Bull. 53: 100-102 (2005); Petasis, N. A. and Zavialov, I. A. J. Am. Chem. Soc. 119: 445-6 (1997); Petasis, N. A. and Zavialov, I. A., J. Am. Chem. Soc. 120: 11798-9; Prakash, G. K. S. et al. Org. Lett. 2: 3173-6 (2000); Prakash, G K S, et al. J. Org. Chem. 67: 3718-23 (2002); the disclosures of all of which are incorporated herein by reference.

General methods of synthesis, especially synthesis of esters are provided in “Comprehensive Organic Transformations” 2^{nd} Edition, Larock, R C, Wiley, New York, 1999 and “Protective Groups in Organic Synthesis”, Greene T and Wuts P G M, Edition 3, Wiley, New York, 1999.

Scheme 1 below illustrates a preparative route for unnatural amino acids reported by Petasis and Zavialov (1997). In this illustration, the amine component is a chiral amine, S-2-phenylglycinol and it exhibits a stereoselective preference for one chiral product. Although this reaction is normally carried out using aldehydes as a substrate, it is known that ketones also yield products with a quarternary center as shown in Scheme 2 (reviewed by Petasis (2005) “Multicomponent Reactions with Organoboron Compounds” In Multicomponent Reactions, pp 199-223, J. Zhu and H Bienayme, Eds., Wiley-VCH Verlag, Weinheim, Germany). In the case of quarternary centers, a less hindered product is obtained if a singly substituted amine is used, such as benzyl amine. This approach can be of benefit to facilitate later reactions such as the dihydroxylation reaction.

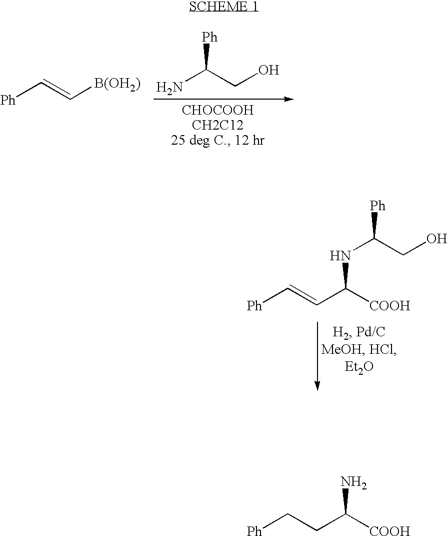

This multicomponent condensation route is readily extended for making test quantities of the compounds of the invention as illustrated below in Scheme 2, wherein W is as defined in Formula (I) and Rj is any of the alkyl or arylalkyl chains required to make the compounds defined in Formula (I). Importantly, specific stereoisomers around the quarternary carbon at position 2 in the final structure can be generated by use of a specific isomer of the 2-phenylglycinol structure which comprises the amino component. This type of reaction sequence has already been extended to the preparation of anti-α-(difluoromethyl)-β-amino alcohols and of (2S,3R)-difluorothreonine (Prakash, G. K. S. et al. J. Org. Chem. 67: 3718-23 (2002). The required vinylboronic acid components are readily prepared from the corresponding alkyne by hydroboration through treatment with catecholborane, followed by hydrolysis (for example—Sugiyama, S. et al. Chem. Pharm. Bull. 53:100-2 (2005)). Many routes are available for production of the pyruvate starting materials, for example from the corresponding amino acids through deamination.

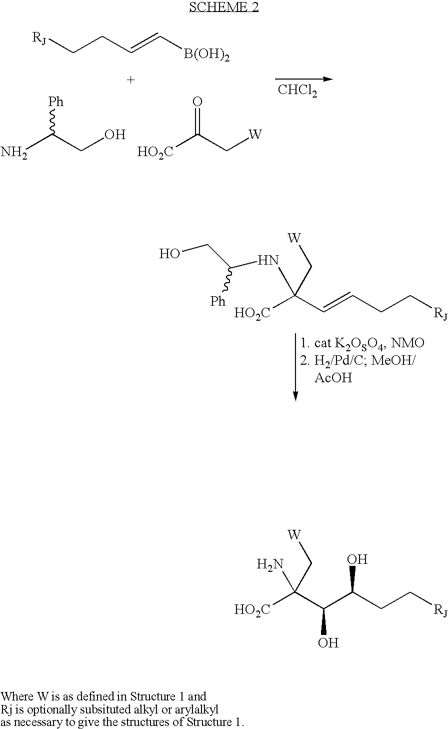

Specific enhancements to the Scheme 2 entail the production of single isomers of the compounds of the invention. For example the introduction of the chiral quarternary center at C2 can be done stereoselectively through the use of chiral amine adducts or chiral boronate esters (Southwood, T. J. et al. Tetrahedron 62: 236-42 (2006)). As shown above, specific chiral amine components can be incorporated to generate single chiral products (Petasis, N. A. and Zavialov, I. A. J. Am. Chem. Soc. 119: 445-6 (1997)). The oxidation step using K₂OsO₄ as preoxidant or OsO₄ and N-methylmorpholine-N-oxide (NMO) alternatively can be carried out using the commercially available asymmetric dihydroxylation reagents AD-mix-α or β (Kolb, H. C., et al. Chem. Rev. 94: 2483-2547 (1994)) to yield syn-hydroxylation but with high diastereoselectivity.

As Scheme 3 illustrates a similar synthetic procedure for preparing of an analog having increased water solubility, analogs of myriocin which contain two hydroxyl functional groups alpha and beta to the head group, can be prepared from native myriocin using a variation of the approach reported by Chen, J K, et al. (1999). Shown below is an exemplary synthetic procedure using starting material modified on the primary hydroxyl position from that reported in Chen et al. to obtain a range of analogs having various functionalities in R3 by employing a Wittig-type reaction with iodoalkyl compounds. For example, R3 can be alkyl, haloalkyl, aryl, aralkyl, and the like. Scheme 3 is a chiral preparation and corresponding enantiomers can be produced using this procedure by protecting the NH/CO₂H functional groups, followed by inversion chemistry on the secondary OH groups. Exemplary compounds are readily prepared from the corresponding iodoalkyl compounds using the procedure illustrated below.

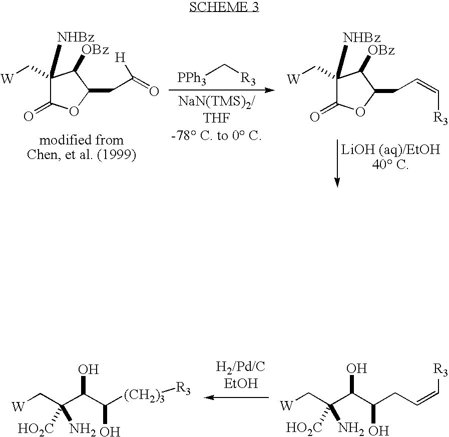

Compounds having a single hydroxyl function alpha to the head group can be prepared in the synthetic method illustrated below in Scheme 4. Similar reagents with different protecting groups may be used to carry out these synthetic steps with greater or lesser yields, depending on the actual substrates used. Exemplary compounds 21, 23, 27, and 29 are prepared from their corresponding starting materials by a route analogous to that shown in Scheme 4.

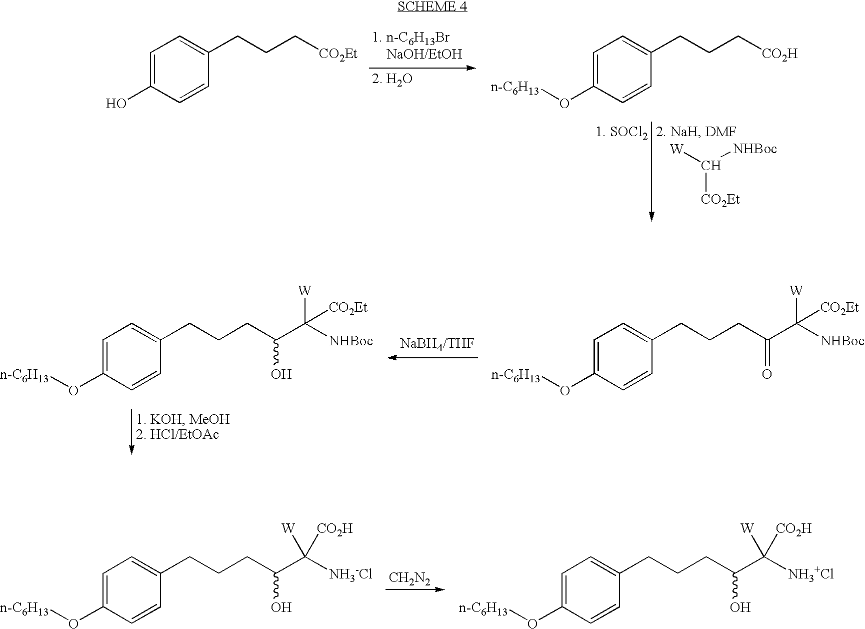

Similarly, compounds with a single hydroxyl function, beta to the serine like head group (e.g. compound 16) are prepared through a route starting from the corresponding, readily available alpha-haloketones or alpha-hydroxyketones according to Scheme 5.

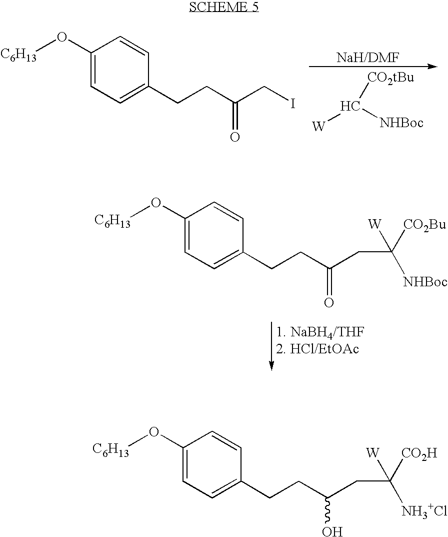

An example of the synthesis of a compound of the invention is given in Scheme 6. This synthesis given is for compound 12 (Example 1) and is an extension of a route used for the synthesis of FTY720 by Sugiyama, S. et al. Chem. Pharm. Bull. 53:100-2 (2005) that uses the Petasis reaction (Petasis, N. A. (2005) “Multicomponent Reactions with Organoboron Compounds” In Multicomponent Reactions, pp 199-223, J. Zhu and H Bienayme, Eds., Wiley-VCH Verlag, Weinheim, Germany).

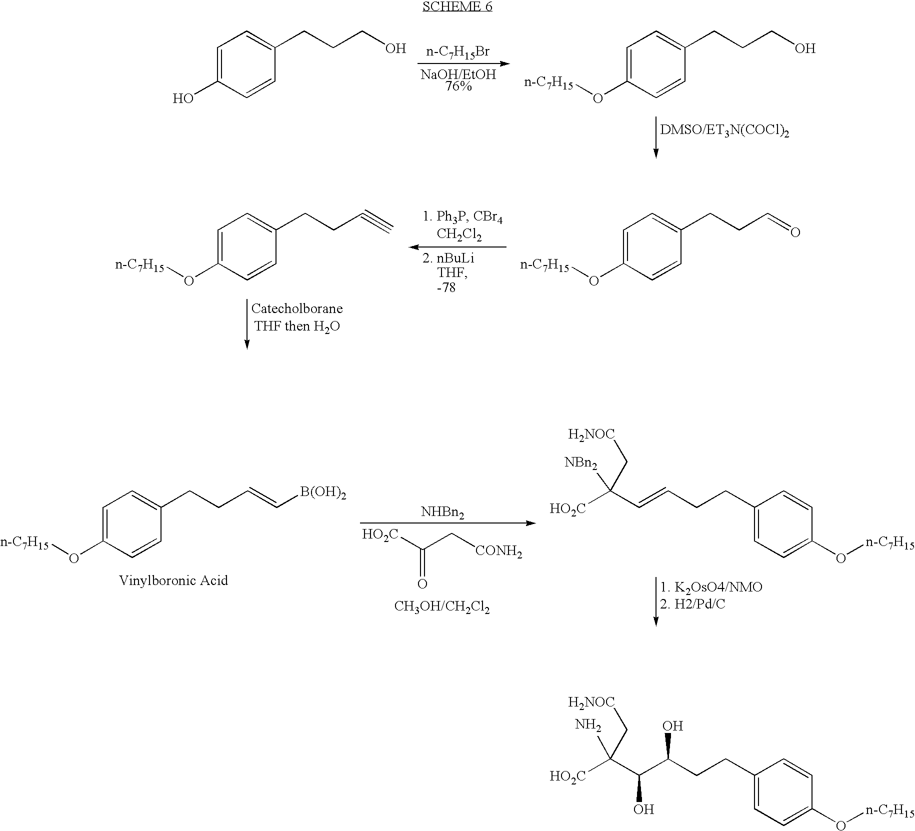

Additional routes to the compounds of the invention are extensions from previous synthetic routes toward the natural products inhibitors of SPT (Liao, J. et al. Tetrahedron 61: 4715-33 (2005)), which are unsuitable as pharmaceutical agents. Thus work by Trost (Trost, B. M. and Lee, C. J. Am. Chem. Soc. 123: 12191-201 (2001)) and Kobayashi (Kobayashi, S, and Furuta, T. J. Am. Chem. Soc. 120: 908-19 (1998) offer a fertile background for the design of syntheses of analogs such as the compounds of the invention. Examples are given in Schemes 7 and 8. Thus in Scheme 7, the illustrated route (Trost, B. M. and Lee, C., 2001), the content of which is incorporated by reference, begins with intermediate 21 from that reference and uses reactions illustrated therein. Although the stereochemistry at position 2 is important, that the stereochemistry at other positions is much less so. Stereochemistry at all positions is easily modified by routes illustrated in this reference. For example, 2-position stereochemistry is inverted by using an amino acid of the opposite configuration to begin the synthesis. Additionally, with reference to the above Trost publication, stereochemistry at position 3 can be inverted by transesterification/saponification of the Ac group, activation to the triflate and inversion by rearrangement of the benzoate group. An attractive feature is that the structure of the quaternary head group derives from that of the corresponding amino acid and there are a host of such structures available for incorporation into SPT compounds of the invention. Essentially any commercially available amino acid (with a wide range of side chains) can be incorporated into this reaction scheme.

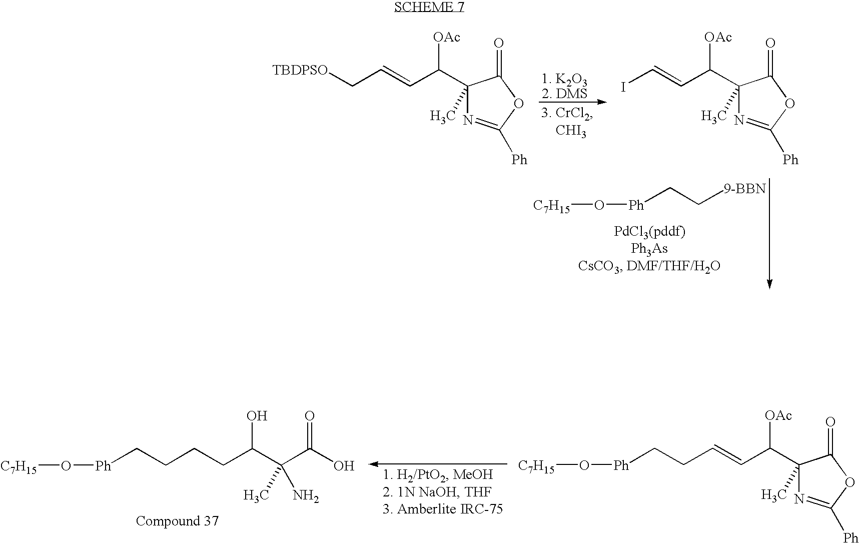

Similarly, as illustrated in Scheme 8, Kobayashi and Furuta J. Am. Chem. Soc. 120: 908-19 (1998) illustrate the use of a very versatile lactim intermediate as devised by Schöllkopf, U. Pure Appl Chem. 55: 1799 (1983). This route provides multihydroxyl analogs (Compound 38) or a saturated alkyl chain, depending on whether reduction or dihydroxylation of the double bond is pursued. The corresponding bislactim reagent generated from D-(R)-Val-Gly-OEt (isoPr rather than CH3 as the chirality determining substituent) was used by Hinterding, et al. (2002) to generate similar alpha methyl amino acid head groups as intermediates and illustrates the use of this route to generate compound of the invention. This route can be used also with aldehydes to generate Compound 38 and analogs thereof. This route again offers great flexibility in the synthesis of analogs, depending on which amino acids are used to build the lactim intermediate.

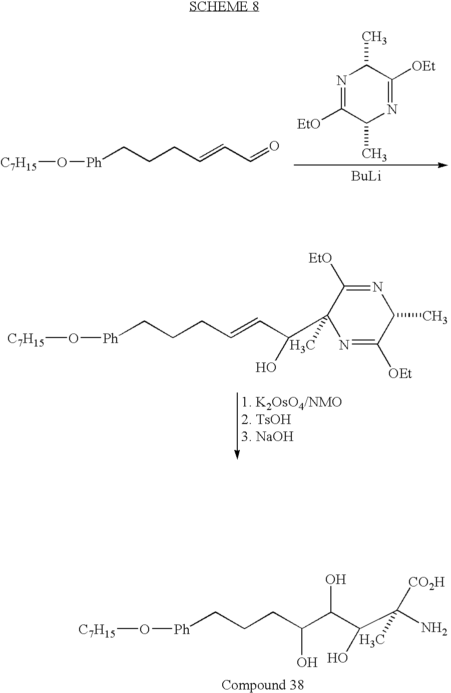

General routes to the synthesis of amino acids can be readily applied to the preparation of the compounds of the invention and some general routes to the synthesis of amino acids have been referenced above (for example, Seebach, D., et al. Helv. Chim. Acta 70: 1194-1216 (1987); Schölkopf, U. Pure Appl. Chem. 55: 1799 (1983)). Additional routes, especially for the incorporation of amine containing W side chains, make use of the Bucherer-Bergs reaction and asymmetric synthesis routes as outlined in Viso, A., et al. Chem. Rev. 105: 3167-96 (2005), the content of which is incorporated by reference.

An example of the use of a diamine intermediate to prepare the compounds with amine containing W side chains referred to above is shown in Scheme 9, wherein asparagine is used to prepare the first chiral intermediate. This is subsequently converted to the desired diamine intermediate by two alkylation steps (see Castellanos, E, et al. Helv. Chim. Acta 87: 1016-24 (2004). The primary amine side chain is the most powerful and least sterically hindered nucleophile and can be modified by many reagents without the need for protection of those functional groups. Thus reaction of the aminomethyl containing product can be reacted with sulfonyl halides or isocyanates to yield products where W═—CH₂—NHSO₂—R or W=—CH₂—NHCOR, respectively. In the case of highly reactive reagents, it is a simple procedure to protect the other functional groups through sequential steps, however, to yield even cleaner products. Either stereochemistry can be introduced at each position of potential chirality.

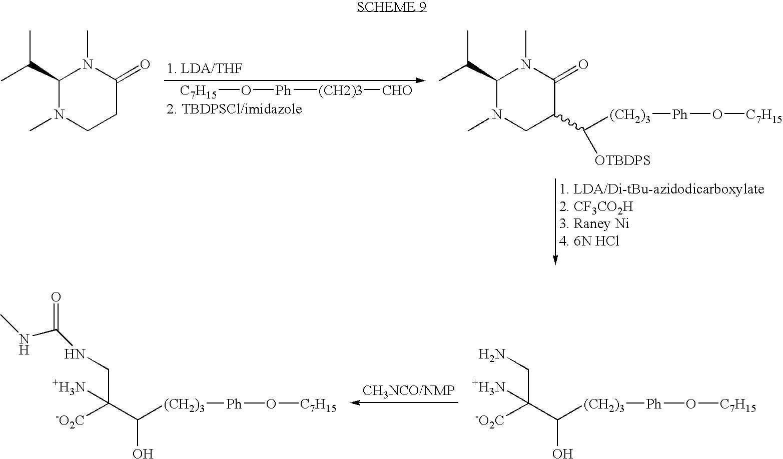

A specific example of the application of the Petasis reaction applied to the synthesis of compounds of the invention is illustrated below in Scheme 10. In this generalizable reaction the amino acid head group is generated in racemic (as shown) or chiral form. Use of chiral amine components yields single enantiomers from the Petasis coupling. For example, use of R or S-2-phenylglycinol yields a single isomer product that can be carried forward through dihydroxylation and deprotection to yield final products.

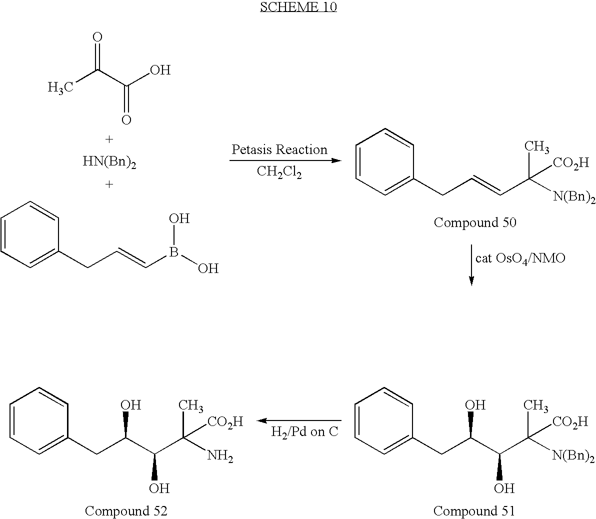

A chiral version of this schema is shown in SCHEME 11, as shown below and in Example 5. Here the approach is illustrated through the use of R-2-phenylglycinol as a chiral reactant, but other similar chiral amines have been used for such reactions and have been tested for this reaction in our hands. 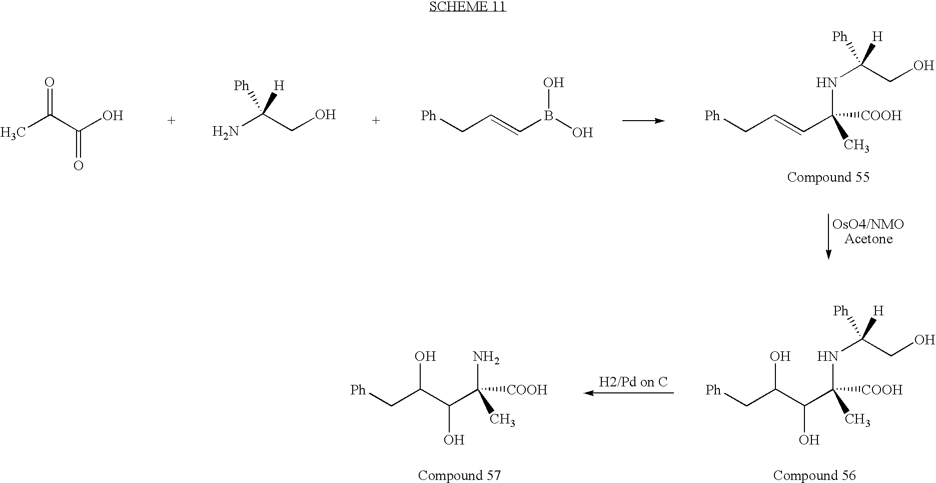
To reiterate, the bis-lactim method offers a chiral synthetic approach to a wide range of the compounds of the invention (SCHEME 12). Incorporation of the W substituent onto the bis-lactim available from R-Valyl-Glycine-OEt (Fluka), followed by acylation, aldol reaction or alkylation, can yield the final products in good yield and in chiral form. Thus compound 60 represents a critical family of intermediates for the production of the compounds of the invention. For example Compound 60, where W═CH2F or CHF2 was prepared by Groth U and Schollkopf, U, Synthesis:673-5 (1983). Similarly the other compounds of the invention where W encompasses a functional group on a carbon atom can be generated from the corresponding alkylating reagent (illustrated in the scheme as having an iodide moiety as the leaving group, but a full range of leaving groups are workable here, from halides to sulfonates and beyond) by reaction with the anion of the bis-lactim formed from R-Valyl-Glycine-OEt. Thus alkylation of the bis-lactim anion with ICH2CH3 yields W=Et; I—CH2CF3 yields W═CH2CF3; Br—CH2CN yields W═CH2CN; Br—CH2-SO2-CH3 yields W═CH2-SO2-CH3; Br—CH2-O—CF3 yields W═CH2-O—CF3; I—CH2-CO2Et yields CH2-CO2Et which can subsequently be converted to the corresponding amides (e.g. W═CH2-CO—NHCH3). From the intermediated W═CH2NH2 the corresponding urethane and urea products are produced by reaction with acylating agents or isocyanates (for example CH3—CO—Cl or CH3NCO). Mild hydrolysis and workup by ion exchange (e.g. see Sano S, et al. (1995) Tetrahedron Lett 36: 2097-2100) yields the products of the invention.
Similarly, the further elaboration of the head group can be done by aldol reaction, acylation or alkylation. For example, use of activated acyl groups such as acid halides was shown to generate the ketone products following hydrolysis (Schollkopf, U., et al. Annalen 1988: 781-6). These ketone products are compounds of the invention, but also offer the opportunity for stereoselective reduction to the corresponding alcohol or diol, as illustrated in SCHEME 12.

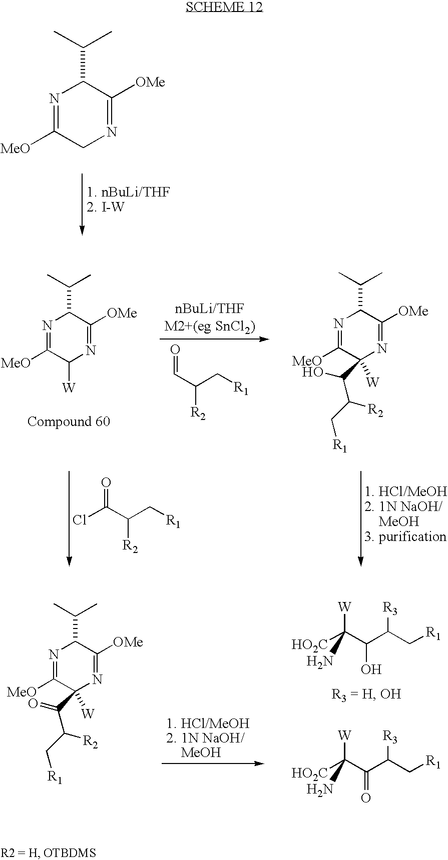

As illustrated in SCHEME 12, there is flexibility in the details of the reaction scheme. The solvent used during the deprotonation/alkylation is typically THF, but the present invention is not limited as such and may be another similar aprotic solvent (e.g. dioxane, glyme or the like) with or without additives for solvation assistance in the reaction (e.g. dipolar aprotic solvents like 1,3-dimethyl-2-imidazolidinone (DMEU) and the like). Addition of coordinating metal salts can lead to different diastereoselectivities. Although SnCl₂ is used here for illustration, experiments on such reactions have been carried out with other divalent salts like MgBr2, ZnCl2 and the like, with varying diastereoselectivities observed at the OH position. The use of SnCl₂ has shown favorable results for a very similar aldol condensation to generate the desired isomer on a route to sphingofungin F (Kobayashi, S., et al. (1998a)) and has been used in our studies.

A specific example of this general approach is outlined in SCHEME 13, below. In this general example, the W group is methyl (introduced as methyl iodide in the bis-lactim alkylation step) and the aldol coupling is performed on 4-phenylbutyraldehyde to yield compound 65. Addition of SnCl₂ to the reaction mixture induces asymmetry in the transition state and thereby enriches the product mixture in a single stereoisomer at the 3 position. Further optimization of reaction solvent, temperature, cosolvents, etc. can yield further improvements in the yield of the desired stereoisomer and are anticipated in this disclosure. The intermediate Compound 65 is converted by mild hydrolysis, purification and conversion to the zwitterion to the desired material, Compound 66. In a similar manner, using the corresponding alkylating agents to give the desired W substituent and the corresponding aldehydes to give the desired aldol product, the compounds of the invention are prepared.

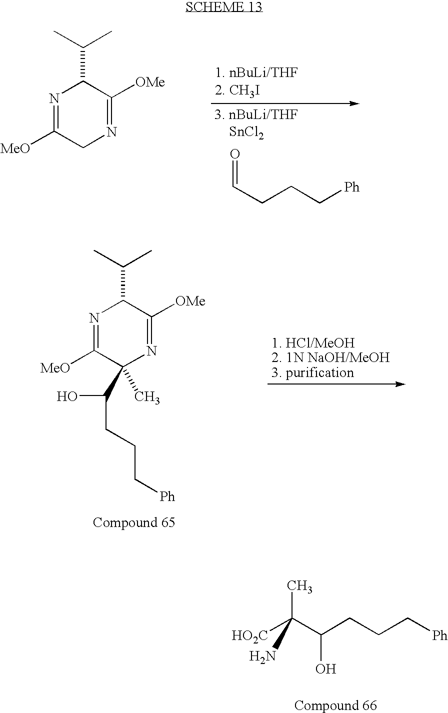

A further specific example of the synthesis of representative members of the compounds of the invention is shown in SCHEME 14. The commercially available mono-TBDMS protected 1,4-butandiol is subjected to Swern oxidation followed by CrCl2-mediated iodomethyleneation to the vinyl iodide in the manner of Takai, et al (1986). In situ reaction of compound 66 with 9-BBN—H followed by reaction of the resulting borane with the vinyl iodide 65 is catalyzed by Pd in a B-alkyl Suzuki reaction to yield the trans-olefin 67 (see Trost, B M and Lee, C, 2001 and references therein for a similar condensation and experimental details). Deprotection by F— and Swem oxidation yielded the aldehyde 68. This aldehyde is reacted with the Li salt of the Schöllkopf bis-lactim derived from R-Valyl-S-alanine methyl ester (also commercially available) as described in SCHEME 12. Equally effective is a two step alkylation/aldol sequence from the R-Valyl-Glycine methyl ester bis-lactim as described in SCHEME 12. In the present case, the first step alkylation is with CH₃I to yield W═CH₃ analogs. Mild acid hydrolysis in aqueous CH3CN yields the product compound 70, which is purified by preparative reversed-phase chromatography on C-18 using a gradient from 0.1% TFA to 100% CH3CN/0.1% TFA.
In a similar manner, using the corresponding vinyl iodides and alkene components, are prepared the aldehyde intermediates to generate the compounds of the invention as shown in this scheme.

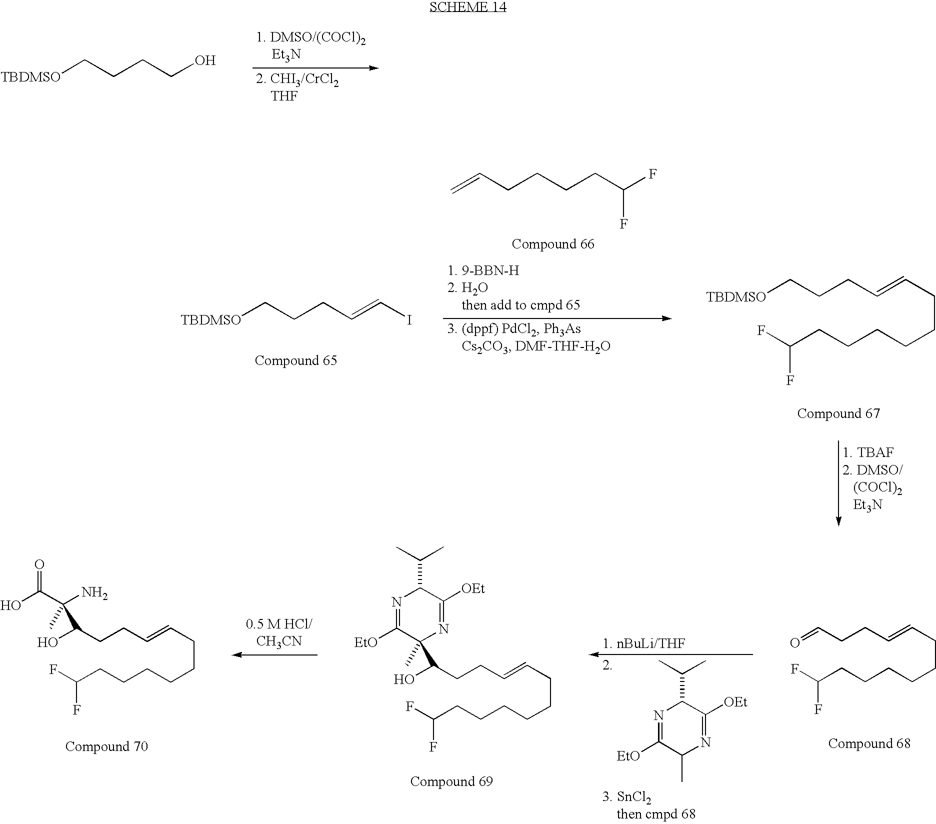

### IV. PHARMACEUTICAL COMPOSITIONS

Compositions presented herein include compounds provided herein and a pharmaceutically acceptable carrier.

A. Formulations

Pharmaceutically useful compositions comprising the compounds of the present invention may be formulated according to known methods such as by the admixture of a pharmaceutically acceptable carrier. Examples of such carriers and methods of formulation may be found in Remington's Pharmaceutical Sciences. To form a pharmaceutically acceptable composition suitable for effective administration, such compositions will contain an effective amount of the compound, e.g., a prodrug or an active species (e.g., the corresponding acid of the ester or prodrug), of the present invention.

Suitable formulations for administering the present compounds include topical, transdermal, oral, systemic, and parenteral pharmaceutical formulations. Compositions containing compounds herein can be administered in a wide variety of therapeutic dosage forms in conventional vehicles for administration. For example, the compounds or modulators can be administered in such oral dosage forms as tablets, capsules (each including timed release and sustained release formulations), pills, powders, granules, elixirs, tinctures, solutions, suspensions, syrups and emulsions, or by transdermal delivery or injection. Likewise, they may also be administered in intravenous (both bolus and infusion), intraperitoneal, subcutaneous, topical with or without occlusion, transdermal, or intramuscular form, all using forms well known to those of ordinary skill in the pharmaceutical arts. The present compounds may be delivered by a wide variety of mechanisms, including but not limited to, transdermal delivery, or injection by needle or needle-less injection means.

B. Dosages

Embodiments include pharmaceutical compositions comprising an effective amount of compounds presented herein. Effective dosages of compounds disclosed herein may be defined by routine testing in order to obtain optimal inhibition of serine palmitoyl transferase while minimizing any potential toxicity.

As is well known to one of skill in the art, effective amounts can be routinely determined and vary according to a variety of factors such as the individual's condition, weight, sex, age, medical condition of the patient, the particular condition to be treated, severity of the condition to be treated, route of administration, renal and hepatic function of the patient, and the particular compound thereof employed. A physician or veterinarian of ordinary skill can readily determine and prescribe the effective amount of the drug required to prevent, counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug.

An effective but non-toxic amount of the compound desired can be employed as a serine palmitoyl transferase-modulating agent. Dosages contemplated for administration of the present compounds range from 0.01 to 1,000 mg per patient, per day. For oral administration, the compositions are preferably provided in the form of scored or un-scored tablets containing 0.01, 0.05, 0.1, 0.5, 1.0, 2.5, 5.0, 10.0, 15.0, 25.0, and 50.0 milligrams of the active ingredient for the symptomatic adjustment of the dosage to the patient to be treated. Dosage amounts may also vary by body weight and can range, for example, from about 0.0001 mg/kg to about 100 mg/kg of body weight per day, preferably from about 0.001 mg/kg to 10 mg/kg of body weight per day.

Compounds may be administered in a single daily dose, or the total daily dosage may be administered in divided doses of two, three, or four times daily. To be administered in the form of a transdermal delivery system, the dosage administration will be continuous rather than intermittent throughout the dosage regimen.

The dosages of the compounds of the present invention are adjusted when combined with other therapeutic agents. Dosages of these various agents may be independently optimized and combined to achieve a synergistic result wherein the pathology is reduced more than it would be if either agent were used alone. In addition, co-administration or sequential administration of other agents may be desirable.

The present invention further comprises physical modes and means (such as, but not limited to, kits) of providing combined administration of the compounds of the present invention and other desired therapeutic agents.

C. Derivatives

Embodiments of compounds presented herein include “chemical derivatives.” Chemical derivatives comprise compounds herein and additional moieties that improve the solubility, half-life, absorption, etc. of the compound. Chemical derivatives may also comprise moieties that attenuate undesirable side effects or decrease toxicity. Examples of such moieties are described in a variety of texts, such as Remington's Pharmaceutical Sciences, and are well known to one of skill in the art.

D. Carriers and Excipients

Compounds herein can be administered in admixture with suitable pharmaceutical diluents, excipients, or carriers (collectively referred to herein as “carrier” materials) suitably selected with respect to the intended form of administration, that is, oral tablets, capsules, elixirs, syrups and the like, and consistent with conventional pharmaceutical practices.

For oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

For liquid forms the active drug component can be combined in suitably flavored suspending or dispersing agents such as the synthetic and natural gums, for example, tragacanth, acacia, methyl-cellulose and the like. Other dispersing agents which may be employed include glycerin and the like.

For parenteral administration, sterile suspensions and solutions are desired. Isotonic preparations which generally contain suitable preservatives are employed when intravenous administration is desired.

Topical preparations comprising the present compounds can be admixed with a variety of carrier materials well known in the art, such as alcohols, aloe vera gel, allantoin, glycerine, vitamin A and E oils, mineral oil, PPG2 myristyl propionate, and the like, to form, for example, alcoholic solutions, topical cleansers, cleansing creams, skin gels, skin lotions, and shampoos in cream or gel formulations.

Compounds can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidyicholines.

Compounds presented herein may also be delivered by the use of monoclonal antibodies as individual carriers to which the compound molecules are coupled. Compounds may be coupled with soluble polymers as targetable drug carriers. Such polymers can include polyvinyl-pyrrolidone, pyran copolymer, polyhydroxypropylmethacryl-amidephenol, polyhydroxy-ethylaspartamideplhenol, or polyethyl-eneoxidepolylysine substituted with palmitoyl residues. Furthermore, compounds may be coupled to biodegradable polymers useful in achieving controlled release of a drug, such as polylactic acid, polyepsilon caprolactone, polyhydroxy butyric acid, polyorthoesters, polyacetals, polydihydro-pyrans, polycyanoacrylates, cross-linked or amphipathic block copolymers of hydrogels, and other suitable polymers known to those skilled in the art.

For oral administration, compounds may be administered in capsule, tablet, or bolus form. The capsules, tablets, and boluses comprise an appropriate carrier vehicle, such as starch, talc, magnesium stearate, or di-calcium phosphate.

Unit dosage forms are prepared by intimately mixing compounds with suitable finely-powdered inert ingredients including diluents, fillers, disintegrating agents, and/or binders such that a uniform mixture is obtained. An inert ingredient is one that will not adversely react with the compounds. Suitable inert ingredients include starch, lactose, talc, magnesium stearate, vegetable gums and oils, and the like. Compounds can be intimately mixed with inert carriers by grinding, stirring, milling, or tumbling.

Injectable formulations comprise compounds herein mixed with an appropriate inert liquid carrier. Acceptable liquid carriers include the vegetable oils such as peanut oil, cottonseed oil, sesame oil and the like as well as organic solvents such as solketal, glycerol formal and the like. As an alternative, aqueous parenteral formulations may also be used. The vegetable oils are the preferred liquid carriers. The formulations are prepared by dissolving or suspending the compound in a liquid carrier.

Topical application of compounds is possible through the use of, for example, a liquid drench or a shampoo containing the instant compounds or in modulators as an aqueous solution or suspension. These formulations may comprise a suspending agent such as bentonite and optionally, an antifoaming agent.

The pharmaceutical oral dosage forms including formulations described herein, which include a compound of Formula (I), can be further formulated to provide a controlled release of the compound of Formula (I). Controlled release refers to the release of the compound of Formula (I) from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery to the lower gastrointestinal tract. In some embodiments the polymers for use in the present invention are anionic carboxylic polymers. In other embodiments, the polymers and compatible mixtures thereof, and some of their properties, include, but are not limited to:

Shellac, also called purified lac, a refined product obtained from the resinous secretion of an insect. This coating dissolves in media of pH>7;

Acrylic polymers. The performance of acrylic polymers (primarily their solubility in biological fluids) can vary based on the degree and type of substitution. Examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;

Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP psuedolatex with particles<1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions;

Poly Vinyl Acetate Phthalate (PVAP). PVAP dissolves in pH>5, and it is much less permeable to water vapor and gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., camuba wax or PEG) may be added to the coatings besides plasticizers to solubilize or disperse the coating material, and to improve coating performance and the coated product.

In other embodiments, the formulations described herein, which include a compound of Formula (I), are delivered using a pulsatile dosage form. A pulsatile dosage form is capable of providing one or more immediate release pulses at predetermined time points after a controlled lag time or at specific sites. Pulsatile dosage forms including the formulations described herein, which include a compound of Formula (I), may be administered using a variety of pulsatile formulations known in the art. For example, such formulations include, but are not limited to, those described in U.S. Pat. Nos. 5,011,692, 5,017,381, 5,229,135, and 5,840,329, each of which is specifically incorporated by reference. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284, all of which are specifically incorporated by reference. In one embodiment, the controlled release dosage form is pulsatile release solid oral dosage form including at least two groups of particles, (i.e. multiparticulate) each containing the formulation described herein. The first group of particles provides a substantially immediate dose of the compound of Formula (I) upon ingestion by a mammal. The first group of particles can be either uncoated or include a coating and/or sealant. The second group of particles includes coated particles, which includes from about 2% to about 75%, preferably from about 2.5% to about 70%, and more preferably from about 40% to about 70%, by weight of the total dose of the compound of Formula (I) in said formulation, in admixture with one or more binders. The coating includes a pharmaceutically acceptable ingredient in an amount sufficient to provide a delay of from about 2 hours to about 7 hours following ingestion before release of the second dose. Suitable coatings include one or more differentially degradable coatings such as, by way of example only, pH sensitive coatings (enteric coatings) such as acrylic resins (e.g., Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, and Eudragit® NE30D, Eudragit® NE 40D®) either alone or blended with cellulose derivatives, e.g., ethylcellulose, or non-enteric coatings having variable thickness to provide differential release of the formulation that includes a compound of Formula (I).

Many other types of controlled release systems known to those of ordinary skill in the art and are suitable for use with the formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983, each of which is specifically incorporated by reference.

E. Modes of Administration

Other factors affecting dosage amounts are the modes of administration. The pharmaceutical compositions of the present invention may be provided to the individual by a variety of routes including, but not limited to subcutaneous, intramuscular, intra-venous, topical, transdermal, oral and any other parenteral or non-parenteral route. Furthermore, compounds can be administered in intranasal form via topical use of suitable intranasal vehicles, or via transdermal routes, using those forms of transdermal skin patches well known to those of ordinary skill in that art.

The compounds or modulators may alternatively be administered parenterally via injection of a formulation consisting of the active ingredient dissolved in an inert liquid carrier. Injection may be either intramuscular, intraruminal, intratracheal, or subcutaneous, either by needle or needle-less means.

F. Pharmaceutically Acceptable Salts

Embodiments include compounds presented herein in the form of a free base or as a pharmaceutically acceptable salt. Exemplary pharmaceutically acceptable salts include hydrobromic, hydroiodic, hydrochloric, perchloric, sulfuric, maleic, fumaric, malic, tartaric, citric, benzoic, mandelic, methanesulfonic, hydroethanesulfonic, benzenesulfonic, oxalic, pamoic, 2-naphthalenesulfonic, p-toluenesulfonic, cyclohexanesulfamic and saccharic. Ion exchange, metathesis or neutralization steps may be used to form the desired salt form.

G. Combinations

Embodiments include compositions comprising compounds presented herein in combination with another active agent. Exemplary active agents which may be employed include insulin, insulin analogs, incretin, incretin analogs, glucagon-like peptide, glucagon-like peptide analogs, exendin, exendin analogs, PACAP and VIP analogs, sulfonylureas, biguanides, a-glucosidase inhibitors, ligands for the Peroxisome Proliferator-Activated Receptors (PPARs) of all classes, inhaled formulations containing bronchodilators, beta 2 adrenoceptor agonists, inhaled corticosteroids, anti-inflammatory steroids, leukotriene modifiers, leukotriene receptor antagonists, chemokine modifiers, chemokine receptor antagonists, cromolyn, nedocromil, xanthines, anticholinergic agents, immune modulating agents, other known anti-asthma medications, phosphodiesterase inhibitors, other known anti-inflammatory medications and the like.

The combination of the compounds of the present invention with inhaled formulations containing bronchodilators, beta 2 adrenoceptor agonists, inhaled corticosteroids, anti-inflammatory steroids, leukotriene modifiers, leukotriene receptor antagonists, chemokine modifiers, chemokine receptor antagonists, cromolyn, nedocromil, xanthines, anticholinergic agents, immune modulating agents, other known anti-asthma medications, phosphodiesterase inhibitors, other known anti-inflammatory medications and the like is of use in the treatment of COPD and emphysema, and related diseases and disorders.

The term “insulin” shall be interpreted to encompass insulin analogs, natural extracted human insulin, recombinantly produced human insulin, insulin extracted from bovine and/or porcine sources, recombinantly produced porcine and bovine insulin and mixtures of any of these insulin products. The term is intended to encompass the polypeptide normally used in the treatment of diabetics in a substantially purified form but encompasses the use of the term in its commercially available pharmaceutical form, which includes additional excipients. The insulin is preferably recombinantly produced and may be dehydrated (completely dried) or in solution.

The terms “insulin analog,” “monomeric insulin” and the like are used interchangeably herein and are intended to encompass any form of “insulin” as defined above, wherein one or more of the amino acids within the polypeptide chain has been replaced with an alternative amino acid and/or wherein one or more of the amino acids has been deleted or wherein one or more additional amino acids has been added to the polypeptide chain or amino acid sequences, which act as insulin in decreasing blood glucose levels. In general, the term “insulin analogs” of the present invention include “insulin lispro analogs,” as disclosed in U.S. Pat. No. 5,547,929, incorporated hereinto by reference in its entirety; insulin analogs including LysPro insulin and humalog insulin, and other “super insulin analogs”, wherein the ability of the insulin analog to affect serum glucose levels is substantially enhanced as compared with conventional insulin as well as hepatoselective insulin analogs which are more active in the liver than in adipose tissue. Preferred analogs are monomeric insulin analogs, which are insulin-like compounds used for the same general purpose as insulin, such as insulin lispro, i.e., compounds which are administered to reduce blood glucose levels.

“Insulin analogs” are well known compounds. Insulin analogs are known to be divided into two categories: animal insulin analogs and modified insulin analogs (pages 716-20, chapter 41, Nolte M. S, and Karam, J. H., “Pancreatic Hormones & Antidiabetic Drugs” In Basic & Clinical Pharmacology, Katzung, B. G., Ed., Lange Medical Books, New York, 2001). Historically, animal insulin analogs include porcine insulin (having one amino acid different from human insulin) and bovine insulin (having three amino acids different from human insulin) which have been widely used for treatment of diabetes. Since the development of genetic engineering technology, modifications are made to create modified insulin analogs, including fast-acting insulin analogs or longer acting insulin analogs.

Several insulin analog molecules have been on the market prior to the filing date of the subject application. For example, Eli Lilly sells a fast-acting insulin analog called “lispro” under the trade name Humalog® and Novo Nordisk sells another fast-acting insulin analog called “aspart” under the trade name NovoLog®. In addition, Aventis sells a long-acting insulin analog called “glargine” under the trade name Lantus® and Novo Nordisk sells another long-acting insulin analog called “detemir” under the trade name Levemir®. Table 41-4 of the article by Nolte and Karam (2001) referenced above illustrates the wide range of types of molecules generically referred to as insulin preparations.

The term “incretin analogs” refers to incretin hormones responsible for the phenomenon of enhanced insulin secretion in the presence of food in the gut and the this action (GLP-1 and GIP) is widely known (e.g. articles referenced in Creutzfeldt, W, “The [pre-] history of the incretin concept”. Regulatory Peptides 128: 87-91 (2005).

The term “glucagon-like peptide analogs” refers to well known analogs of Glucagon-Like Peptide (GLP1) (e.g. Nourparvar, A., et al. “Novel strategies for the pharmacological management of type 2 diabetes” Trends in Pharmacological Sciences 25, 86-91 (2004)), and reviews of the area discussed their range of structure and function in detail (cf Table 1 in Knudsen, L. B. “Glucagon-like Peptide-1: The Basis of a New Class of Treatment for Type 2 Diabetes”. J. Med. Chem. 47: 4128-4134 (2004) and references therein). Examples of “glucagon-like peptide analogs” include Liraglutide, Albugon, and BIM-51077.

The term “exendin analogs” refers to exendin (also known as exendin-4, exanetide, Byetta®) and its analogs which have been major diabetes research objectives (c.f. Thorkildsen C. “Glucagon-Like Peptide 1 Receptor Agonist ZP10A Increases Insulin mRNA Expression and Prevents Diabetic Progression in db/db Mice”. J. Pharmacol. Exptl. Therapeut. 307: 490-6 (2003)). Exendin is known to be a specific type of glucagon-like peptide-1 mimic. For example, ZP-10 (AVE-010) is an exendin analog that binds to the GLP1 receptor.

The term “PACAP analogs” refers to well known neuromodulator PACAP and its analogs which are important to physiological insulin secretion (c.f. Filipsson, K. et al. “Pituitary Adenylate Cyclase Activating Polypeptide Stimulates Insulin and Glucagon Secretion in Humans”. J. Clin. Endocrinol. Metab. 82: 3093-8 (1997)). PACAP analog synthesis protocols were available and published in the literature (c.f. Yung, S. L., et al. “Generation of Highly Selective VPAC2 Receptor Agonists by High Throughput Mutagenesis of Vasoactive Intestinal Peptide and Pituitary Adenylate Cyclase-activating Peptide”. J. Biol. Chem. 278: 10273-81 (2003); Tsutsumi M., et al. “A Potent and Highly Selective VPAC2 Agonist Enhances Glucose-Induced Insulin Release and Glucose Disposal”. Diabetes 51: 1453-60 (2002)).

The term “VIP analogs” refers to Vasoactive Intestinal Polypeptide (VIP) and its analogs which are homologous molecules to PACAP that bind to the same target receptor, VPAC2. The analogs referred to as PACAP analogs in Tsutsumi et al (2002) and Yung, et al (2003) above also are considered to be VIP analogs. For example, VIP analogs that bind to the VPAC2 receptor include Bay 55-9837 (Tsutsumi et al (2002), above) and Ro 25-1553 (O'Donnell, et al. “Ro 25-1553: A Novel, Long-Acting Vasoactive Intestinal Peptide Agonist. Part 1: In vitro and In Vivo Bronchodilator Studies” J. Pharmacol. Exptl. Therap. 270: 1282-8 (1994)).

The term “sulfonylureas” refers to well known sulfonylureas used for many years in the treatment of type 2 diabetes. Extensive clinical trial literature and reviews of sulfonylureas are available (c.f. Buse, J., et al. “The effects of oral anti-hyperglycaemic medications on serum lipid profiles in patients with type 2 diabetes”. Diabetes Obesity Metabol. 6: 133-156 (2004)). In table 1 in the Buse reference, the major sulfonylureas/glinides are listed chronologically as Glipizide, Gliclazide, Glibenclamide (glyburide), Glimepiride. The last two members of the list (Repaglinide, and Nateglinide) differ in their specific mechanism of action (Meglitinides), but again are oral agents that stimulate insulin secretion. The Buse reference focuses on studies that are directed at lipid effects, but also illustrates classes of compounds well known as “sulfonylureas”. For example, it is widely believed that only a few compounds constitute the major market share of “sulfonylureas,” such as Dymelor, Diabinese, Amaryl, Glucotrol, Micronase, Tolinase, Orinase and their generic equivalents (see pgs 725-32, chapter 41, Nolte M. S., and Karam, J. H., “Pancreatic Hormones & Antidiabetic Drugs” In Basic & Clinical Pharmacology, Katzung, B. G., Ed., Lange Medical Books, New York, 2001).

The term “biguanides” refers to well known biguanides compounds, such as extensively reviewed on pages 716-20, chapter 41, Nolte M. S. and Karam, J. H., “Pancreatic Hormones & Antidiabetic Drugs” In Basic & Clinical Pharmacology, Katzung, B. G., Ed., Lange Medical Books, New York, 2001. For example, well known compounds that constitute the major market share of “biguanides” include metformin (Glucophage), buformin, and phenformin (Buse, J., et al. “The effects of oral anti-hyperglycaemic medications on serum lipid profiles in patients with type 2 diabetes.” Diabetes Obesity Metabol. 6: 133-156 (2004)).

The term “α-glucosidase inhibitors” refers to well known compounds having α-glucosidase inhibitors activity which has been the subject of extensive clinical studies (pg 729-30, chapter 41, Nolte M. S., and Karam, J. H., “Pancreatic Hormones & Antidiabetic Drugs” In Basic & Clinical Pharmacology, Katzung, B. G., Ed., Lange Medical Books, New York, 2001; Buse, J., et al. “The effects of oral anti-hyperglycaemic medications on serum lipid profiles in patients with type 2 diabetes.” Diabetes Obesity Metabol. 6: 133-156 (2004)). Compounds that constitute the major market share of “α-glucosidase inhibitors” include acarbose (Precose) and miglitol (Glycet).

The term “Acetyl-CoA Carboxylase inhibitors” refers to well known compounds as reviewed in Harwood, H. J., Jr. “Acety-CoA Carboxylase inhibition for the treatment of metabolic syndrome”. Curr. Opin. Invest. Drugs 5: 283-9 (2004) for this developing area of research as a treatment for the metabolic syndrome, of which type 2 diabetes is a major component.

The term “caspase inhibitors” refers to well know compounds as reviewed in Reed, J. C. “Apoptosis-Based Therapies”. Nature Rev. Drug Disc. 1: 111-121 (2002); Talanian, R. V. and Allen, H. J. “Roles of Caspases in Inflammation and Apoptosis: Prospects as Drug discovery Targets” In Annual Reports in Medicinal Chemistry 33: 273-82, J. A. Bristol, Ed., Academic Press, New York (1998)). Compounds that constitute the major market share of “caspase inhibitors” include VX-765 (Vertex Pharmaceuticals) and IDN-6556 (Idun Pharmaceuticals; Hoglen, N. C., et al. “Characterization of IDN-6556 (3-{2-(2-tert-Butyl-phenylaminooxalyl)-amino]-propionylamino}-4-oxo-5-(2,3,5,6-tetrafluoro-phenoxy)-pentanoic Acid): a Liver-Targeted Caspase Inhibitor”. J. Pharmacol. Exptl. Therapeut. 309:634-40 (2003)), both of which are in clinical trials and may be effective broadly for inflammatory diseases, of which diabetes is a member.

The term “PPAR ligands” refers to compounds having Peroxisome Proliferator-Activated Receptor Ligand activity, also interchangeably referred to as thizolidinediones for the predominant structural class, as compounds active in the treatment of type 2 diabetes (c.f. pg 728, chapter 41, Nolte M. S., and Karam, J. H., “Pancreatic Hormones & Antidiabetic Drugs” In Basic & Clinical Pharmacology, Katzung, B. G., Ed., Lange Medical Books, New York, 2001; Lee, et al. “Minireview: Lipid Metabolism, Metabolic Diseases, and Peroxisome Proliferator-Activated Receptors”. Endocrinol. 144: 2201-7 (2003)). PPAR ligands such as pioglitazone are known to have beneficial effects on protection of pancreatic islets (Diani, A. R., et al. “Pioglitazone preserves pancreatic islet structure and insulin secretory function in three murine models of type 2 diabetes”. Am. J. Physiol. Endocrinol. Metab. 286: E116-122 (2004). Compounds that constitute the major market share of “PPAR ligands” include pioglitizone (Actos) and rosiglitazone (Avandia) (c.f. pg 732 in Nolte, M. S., and Karam, J. H.2001, referenced above). Additional PPAR ligands are undergoing clinical trials.

For combination treatment with more than one active agent, where the active agents are in separate dosage formulations, the active agents can be administered concurrently, or they each can be administered at separately staggered times.

The dosages of the compounds of the present invention are adjusted when combined with other therapeutic agents. Dosages of these various agents may be independently optimized and combined to achieve a synergistic result wherein the pathology is reduced more than it would be if either agent were used alone. In addition, co-administration or sequential administration of other agents may be desirable.

H. Kits

In a preferred embodiment, compounds herein are packaged in a kit. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of Formula (I) compound can consist of one tablet or capsule while a daily dose of the second compound can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory aid, so as to further facilitate compliance with the regimen. An example of such a memory aid is a mechanical counter which indicates the number of daily doses that has been dispensed. Another example of such a memory aid is a battery powered microchip memory coupled with a liquid crystal readout, or audible reminder signal which, for example, reads out the date that the last daily dose has been taken and/or reminds one when the next dose is to be taken.

### V. METHODS OF TREATMENT

An important feature of the present invention relates to the involvement of ceramide as a signaling molecule in inflammatory processes. In addition to its effect on the apoptosis of beta cells relevant to T2D, de novo ceramide can have broader apoptotic effects in human health. Influencing the levels of ceramide can lead to novel treatments of human islets, or islets from other commercially or medicinally important sources, in culture during isolation for transplant with the intent of improving survival of islets in vitro and post transplant. SPT inhibitors can be added to currently used or accepted treatment protocols in order to inhibit, either alone and/or in a synergistic fashion, the loss of islets and beta cells due to apoptotic and/or necrotic processes.

Such basic protocols to be improved on are described in Beattie, et al. 2000 (above, and references therein) and in publications describing the “Edmonton Protocol” (Ryan E A, et al. (2001). Diabetes 50:710-9., and references therein). These protocols may involve the addition of trehalose cryoprotectant and removal of Arg (Beattie G M, et al. (1997). Diabetes 46:519-23), fetal bovine serum, transferrin, selenium (Matsumoto, S. et al. (2003)), or various caspase inhibitors such as Z-VAD-FMK and B-D-FMK (Sauerwald, T. M., et al. 2003); Yang, B., et al. 2004), nicotinamide, sodium butryrate (Otonkoski, T., et al. 1999), caerulein, IBMX (Ohgawara, H., et al., 1991), IGF-II (Ilieva, A., et al. 1999), and the like.

Blockade of de novo ceramide synthesis shows a synergistic improvement in cell survival when comprising addition of compounds of the present invention, e.g., SPT inhibitors, to the protocols enumerated above, and their like. Loss of pancreatic islets in Type 1 Diabetes also shows evidence of inflammatory processes leading to apoptosis and necrosis.

Embodiments of the invention include methods for treating developing Type 1 Diabetes and/or the further loss of islets following transplantation (human or xenobiotic islet cell transplantation) comprising the addition of compounds of the present invention, e.g., SPT inhibitors, to current treatment protocols (IUBMB Life. 2004 July; 56: 387-94. Protecting pancreatic beta-cells. Pileggi A, Fenjves E S, Klein D, Ricordi C, Pastori R L.). Xenobiotic cells contemplated for use in the methods of the present invention include, but are not limited to, porcine, bovine, murine, and other mammalian cell types. The inhibition of de novo ceramide synthesis shows beneficial effects when used alone or as an addition to existing protocols. Such treatment may commence immediately upon detection of loss of beta cell mass or function, and be used alone or in conjunction with immunosuppressive regimens (cyclosporine, mycophenolic acid agents, FTY720, and the like, for example). This is a broadly based mechanism to protect beta cells from a wide array of insults that result in apoptosis and necrosis.

In additional embodiments of this invention, the compounds of the invention are used for the blockade of apoptosis of neuronal cells following spinal injury, and in loss of CNS neurons, e.g. in Alzheimer's disease or stroke. This treatment with an inhibitor of SPT may be used effectively alone or in combination with other treatments such as antioxidants, caspase inhibitors (Benjamins J A et al. Neurochem Res. 28:143-52 (2003)) and/or other treatments for protection from the late effects of stroke that are well known to those skilled in the art.

Compounds and compositions presented herein may be administered to patients in the treatment of a variety of diseases. Preferably, methods of treatment presented herein are directed to patients (i.e., humans and other mammals) with disorders or conditions associated with the activity or hyperactivity of serine palmitoyl transferase (SPT). Accordingly, methods of treating insulin resistance and cardiomyopathy are provided. Compounds effective in treating cardiomyopathy may interfere with the process of cardiomyopathy development. Compounds of the invention may also be used to treat cachexia and sepsis.

Preferred compounds employed in methods of treatment possess desirable bio-availability characteristics. Exemplary compounds are esters which can function as a pro-drug form having improved solubility, duration of action, and in vivo potency. Preferred compounds employed in treatment methods exhibit improved solubility in water and less potential to cross the blood brain barrier to cause side effects, such as altered feeding behavior.

Pharmaceutical compositions are administered to an individual in amounts sufficient to treat or diagnose disorders in which modulation of serine palmitoyl transferase activity is indicated. Examples of diseases or conditions known to be, or suspected of being mediated by serine palmitoyl transferase include, but are not limited to, insulin resistance, type 2 diabetes and its complications, obesity, pro thrombotic conditions, myocardial infarction, congestive heart failure, hypertension, dyslipidemia, and other manifestations of the commonly accepted “Metabolic Syndrome” and “Syndrome X.” Compounds effective in treatment methods herein potently and specifically modulate the enzyme Serine Palmitoyl Transferase.

Furthermore, the anti-inflammatory activity of the compounds of the invention makes them outstanding agents for the treatment or prevention of restenosis by either systemic administration at the time of or prior to PCI or from drug eluting devices such as stents.

It is to be understood that the above description is intended to be illustrative and not restrictive. The scope of the invention should, therefore, be determined not with reference to the above description, but instead with reference to the appended claims along with the full scope of equivalents thereto.

### EXAMPLES

In order to illustrate the invention the following examples are included. These examples do not limit the invention. They are meant to illustrate only exemplary methods and compounds presented herein. Those knowledgeable in chemical synthesis and the treatment of serine palmitoyl transferase related disorders may find other methods of practicing the invention. However those methods are deemed to be within the scope of this invention.

### Example I

### Synthesis of Methyl Ester of Compound 12

In a round-bottomed flask, 500 mL of MeOH is cooled to −5° C. and treated with 0.11 mol of SOCl₂ in a dropwise fashion with stirring. Powdered compound 12 (0.1 mol) is added immediately with cooling and stirring. The solution is allowed to warm slowly to room temperature over a period of 2 hrs. Evaporation of the excess MeOH provides the desired compound (R₁=Me) as the HCl salt in high yield as a white powder. Recrystallization from a suitable solvent (MeOH/Et₂O) provides the desired compound in high purity as a white, waxy solid. In a like manner, additional ester forms of compound herein can be prepared.

### Example 2

### Synthesis of Ethyl Ester of Compound 12

In a round-bottomed flask, 500 mL of EtOH is treated with 0.01 mol of HCl in EtOAc and powdered compound 12 (0.1 mol) is added immediately with cooling and stirring. The solution is warmed to reflux and heated for a period of 24 hrs. Evaporation of the excess EtOH provides the desired compound (R₁=Me) as the HCl salt in high yield as a white powder. Recrystallization from a suitable solvent (EtOH/Et₂O) provides the desired compound in high purity as a colorless oil which slowly forms a waxy solid. In a like manner, additional ester forms of compound herein can be prepared. Alternatively, addition of an equivalent amount of HCl and H2SO4 in EtOH and refluxing for 2 days provides a high yield of product.

### Example 3

### Synthesis of Compound 12

Compound 12 is prepared using the route outlined in Scheme 6, starting with 4-(3-hydroxypropyl)phenol (Aldrich Chemical Company) and initially following the procedure of Kiuchi, M., et al (2000). Compound 12 is obtained as an off white solid and of broad melting point.

### Example 4

### Synthesis of Compound 52

To a solution of pyruvic acid (880 mg, 0.695 mL, 10.0 mmol) in dichloromethane (10 mL) was added dibenzyl amine (1.973 g, 1.92 mL, 10.0 mmol) and trans-3-phenyl-1-propen-1-yl boronic acid (1.62 g, 10.0 mmol). After the reaction mixture was stirred at r.t. for 4 days, it was absorbed on silica gel and purified by silica gel column chromatography (2×20 cm, EtOAc:Hex 1:3-1:2 to give compound 50 as a colorless viscous oil (0.98 g, 20%). ¹H NMR (500 MHz, CDCl₃) δ 9.71 (br, s, 1H), 7.40 (m, 1H), 7.28-7.26 (m, 3H), 7.22-7.18 (m, 10H), 7.12 (m, 2H), 6.03 (dt, J=15.7, 6.8 Hz, 1H), 5.78 (d, J=15.8 Hz, 1H), 4.02-3.90 (m, 4H), 3.34-3.33 (m, 2H), 1.56 (s, 3H). ESIMS (MH+) m/z 386.7.

The alkene, compound 50 (1.25 g, 3.25 mmol), is dissolved in acetone (10 mL) and NMO (0.457 g, 3.9 mmol) is added. After water (2 mL) is added, OsO₄ (2 crystals) is added and stirred at r.t. for 3 days. Additional OsO₄ (2 crystals) is added and stirring continued at r.t. for a week. Na₂SO₃ (2.0 g) is added and stirred at r.t. for 1 h. The solution is dried over Na₂SO₄, filtered, and absorbed on to silica gel. It is purified by silica column chromatography (2×25 cm, EtOAc: Hex 1:3-1:0) to give compound 51 as a mixture of diastereomers (two product pools) as white solids, 250 mg (18%) and 290 mg (21%), respectively.

To a solution of the protected compound 51 (150 mg, 0.36 mmol) in MeOH (5.0 mL) and HCl (0.036 mL, 37%, 0.36 mmol) is added Pd/C (20 mg, 10%). The reaction flask is degassed and the reaction mixture is stirred under hydrogen at r.t. overnight. It is filtered through celite, and washed with MeOH (3×5 mL). It is concentrated and purified by reversed-phase HPLC on C-18 packing and using a gradient from 0.1% TFA to 100% 0.1% TFA in CH3CN. Solvent is removed from the pooled peak and the residue is dried to give compound 57 as a mixture of diasteromers as white hygroscopic solid (40 mg, 46%).

In a like manner, substituting the corresponding boronic acids, are prepared the compounds of the invention.

### Example 5

### Synthesis of Compound 57

Pyruvic acid (0.139 mL, 176 mg, 2.0 mmol), (R)-(−)-2-phenylglycinol (274.36 mg, 2.0 mmol) and trans-3-phenyl-1-propen-1-yl boronic acid (324 mg, 2.0 mmol) were dissolved in CH₂Cl₂ (10 mL) and stirred at r.t. under Ar. After the reaction mixture was stirred at r.t. for 4 days, the solvent was removed and it was purified by silica gel column chromatography (1×15 cm, CH—₂Cl₂: MeOH 98:2-92:8) to give compound 55 as a white solid (0.14 g, 21.5%). ¹H NMR (500 MHz, CDCl₃) δ 7.43-7.07 (m, 10H), 5.39, (s, 1H), 5.11 (s, 1H), 4.37 (s, 1H), 3.99 (s, 1H), 3.76 (s, 2H), 1.25 (s, 3H). ESIMS (M-H—) m/z 324.7.

To a solution of the alkene (0.325 g, 1.0 mmol), compound 55, in acetone (5.0 mL) is added water (1.0 mL), NMO (138 mg, 1.2 mmol), and an OsO₄ solution (0.063 mL, 4%, 2.54 mg, 0.01 mmol) in water. After the reaction mixture is stirred at r.t. for 3 days, Na₂SO₃ (0.5 g) is added and stirred at r.t. for 1 h. It is dried over Na₂SO₄, filtered, washed with acetone (3×5 mL) and absorbed on to silica gel. It is purified by silica column chromatography (1×25 cm, CH₂Cl₂: MeOH 98:2-90:10) to give the product, compound 56, as white solid, in low yield.

To a solution of the protected compound 56 (50 mg, 0.14 mmol) in MeOH (3.0 mL) and HCl (0.014 mL, 37%, 0.14 mmol) is added Pd/C (10 mg, 10%). The reaction flask is degassed and the reaction mixture is stirred under hydrogen at r.t. over night. It is filtered through celite, and washed with MeOH (3×5 mL). It is concentrated and purified by reversed-phase HPLC on C-18 packing and using a gradient from 0.1% TFA to 100% 0.1% TFA in CH3CN. Solvent is removed from the pooled peak and the residue is dried to give compound 57 as a white hygroscopic solid (20 mg, 60%).

In a like manner, substituting the corresponding boronic acids, are prepared the compounds of the invention.

### Example 6

### Synthesis of Compound 66

DMSO (1.95 g, 1.64 mL, 25.0 mmol) was added dropwise to a solution of oxalyl chloride (1.52 g, 1.05 mL, 12.0 mmol) in CH₂Cl₂ at −78° C. After stirring at −78° C. for 15 min, 4-phenyl-1-butanol (1.50 g, 1.52 mL, 10.0 mmol) was added dropwise. After 15 min, triethylamine (5.05 g, 6.96 mL) was added dropwise. After warming to r.t. over 2 hours, water (50 mL) was added and the solution was extracted with CH₂Cl₂ (4×50 mL). The combined organic phase was washed with HCl (0.25 N, 3×50 mL), water (50 mL), saturated NaHCO₃, and brine. The organic extract was dried over Na₂SO₄, concentrated, and passed through a short silica column (2×3 cm), eluted with CH₂Cl₂. Solvent was removed to give 4-phenylbutanal as a colorless oil (1.30 g, 87%). ¹H NMR (500 MHz, CDCl₃) δ 9.76 (t, J=1.5 Hz, 1H), 7.31-7.27 (m, 2H), 7.22-7.17 (m, 2H), 2.67 (t, J=7.5 Hz, 2H), 2.46 (dt, J=1.6, 7.32 Hz, 2H), 1.97 (m, 2H).

To a solution of the bislactim (R)-2-isopropyl-3,6-dimethoxy-2,5-dihydropyrazine (1.842 g, 1.792 mL, 10.0 mmol) in THF (20 mL) at −78° C. was slowly added n-BuLi (6.875 mL, 1.6 M in hexanes, 11.0 mmol) under Ar. After stirring at −78° C. for 20 min, A methyl iodide solution (1.562 g, 0.686 mL, 11.0 mmol) in THF (5 mL) was added. It was stirred at −78° C. for 2 h. A further portion of n-BuLi (6.25 mL, 1.6 M in hexanes, 10.0 mmol) was added at −78° C. and the solution was warmed to 0° C. A solution of SnCl₂ (1.0 g, 5.3 mmol) in THF (20 mL) was added and stirred at 0° C. for 15 min. After cooling to −78° C., a 4-phenylbutanal solution (0.74 g, 5.0 mmol) was added. After the mixture was stirred at −78° C. for 3 h, it was warmed to r.t. and a phosphate buffer (pH 7.0, 0.10 M, 150 mL) was added with stirring for an additional 10 min. The mixture was filtered and the solid washed with ether. The aqueous phase was extracted with ether (3×50 mL) and the combined organic phase was washed with brine and dried over Na₂SO₄. Solvent was removed and the residue was purified by silica gel column chromatography (3×25 cm, hexanes: EtOAc 9:1) to give compound 65 as a colorless viscous oil (850 mg, 49%). ¹H NMR (500 MHz, CDCl₃) δ 7.28-7.25 (m, 2H), 7.19-7.15 (m, 3H), 3.94 (d, J=3.5 Hz, 1H), 3.70 (s, 3H), 3.64 (s, 3H), 3.59 (dt, J=2.0, 10.3 Hz, 1H), 2.66-2.60 (m, 2H), 2.28-2.25 (m, 1H), 1.99 (d, J=10.0 Hz, 1H), 1.95-1.85 (m, 1H), 1.75-1.65 (m, 1H), 1.60-1.52 (m, 1H), 1.35 (s, 1H), 1.33-1.23 (m, 1H), 1.08 (d, J=7.0 Hz, 3H), 0.69 (d, J=6.9 Hz, 1H). ESIMS (MNa⁺) m/z 369.4.

Compound 65 (692 mg, 2.0 mmol) is dissolved in acetonitrile (24 mL) containing HCl (0.50 M, 24 mL). After the mixture is stirred at r.t. for 12 h, the solvent is removed, the residue is dissolved in a NaOH solution (2.0 M, 20 mL) in MeOH (20 mL), and the progress of the reaction is monitored by TLC. The reaction mixture was neutralized with a solution of HOAc, and solvent is removed. The residue is purified by reverse phase HPLC to give desired product, compound 66, as an amorphous white solid.

In a like manner, using the corresponding aldehydes, are prepared the compounds of the invention.

### Example 7

### Beta Cell Apoptosis Assay

Rat Pancreatic Islets.

Biological assays are performed as according to Shimabukuro et al. (J. Biol. Chem., 273: 32487-90 (1998)) with certain modifications. Zucker Diabetic Fatty rats are treated for 2 weeks by i.p. injection with compounds presented herein. Pancreatic islets are isolated and the degree of apoptosis is evaluated by electrophoresis. A significant degree of protection is noted for the treated rats in comparison to the control rats. This protection demonstrates that de novo synthesis of ceramide through the SPT pathway is inhibited specifically and results in protection of beta cells from apoptosis.

Human Pancreatic Islets.

An alternative assay for the detection of beta cell apoptosis is performed according to Maedler, K, et al. (2003). Diabetes 52, 726-33). In this assay, incubation with elevated palmitic acid or elevated glucose causes increased apoptosis and protective effects of inhibitors of ceramide synthase exhibit beneficial effects. Results from this assay demonstrate the beneficial effects of the present compounds to inhibit de novo ceramide synthesis at a different, earlier point in the enzymatic pathway, such as inhibition of SPT.

(A) Islet Isolation and Culture—

Islets are isolated from pancreata of organ donors, as described in Oberholzer J, et al. (Transplantation 69: 1115-1123 (2000)). The islet purity is >95% which is determined by dithizone staining. When this degree of purity is not primarily achieved by routine isolation, islets are handpicked. The donors are typically heart-beating cadaver organ donors without a previous history of diabetes or metabolic disorders.

As reported by Maedler et al. (2003), for long-term in vitro studies, the islets are cultured on extracellular matrix-coated plates derived from bovine corneal endothelial cells (Novamed, Jerusalem, Israel), and the cells are allowed to attach to the dishes and spread, to preserve their functional integrity. The contamination by ductal cells after 4 days in culture is estimated to be between 5 and 15%, but almost all ductal cells are found in the periphery of the islets and do not co-localize with β-cells. Islets are cultured in CMRL 1066 medium containing 100 units/ml penicillin, 100 μg/ml streptomycin, and 10% FCS (Gibco, Gaithersburg, Md.), hereafter referred to as culture medium.

Two days after plating, when most islets are attached and begin to flatten, the medium is changed to culture medium containing 5.5 or 33.3 mmol/l glucose supplemented with or without fatty acids (Sigma Chemical, St. Louis, Mo.; palmitic acid [16:0], palmitoleic acid [16:1], oleic acid [18:1], or a mixture of fatty acids [16:0/16:1, 16:0/18:1]). Fatty acids are dissolved at 10 mmol/L in culture medium containing 11% fatty acid-free BSA (Sigma) under nitrogen atmosphere, are shaken overnight at 37° C., are sonicated for 15 min, and are sterile filtered (stock solution). For control experiments, BSA in the absence of fatty acids is prepared, as described above. The effective FFA concentration may be determined after sterile filtration with a commercially available kit (Wako chemicals, Neuss, Germany). The calculated concentrations of non-albumin-bound FFA is derived from the molar ratio of total FFA (0.5 mmol/l) and albumin (0.15 mmol/l) using a stepwise equilibrium model reported in Spector A A et al., Biochemistry 10: 3226-32 (1971). Unbound concentration of palmitic, palmitoleic, and oleic acids are of 0.832, 0.575, and 2.089 micromol/L, respectively, for a final concentration of 0.5 mmol/L FFA. In some experiments, islets are cultured with or without 15 micromol/L C2-ceramide, 15 micromol/L C2-Dihydroceramide (Biomol, Plymouth Meeting, Pa.), 15 micromol/L fumonisin B1 (Sigma), or tested compounds at various concentrations from 10 nmol/L to 100 micromol/L. All of them are first dissolved in prewarmed 37° C. DMSO (Fluka, Buchs, Switzerland) at 5 mmol/L. For control experiments, islets are exposed to solvent alone (0.3% DMSO).

(B) Cell apoptosis—

As reported by Maedler, et al. (2003), the free 3-OH strand breaks resulting from DNA degradation are detected by the terminal deoxynucleotidyl transferase-mediated dUTP nick-end labeling (TUNEL) technique (Gavrieli Y, et al. (1992). J. Cell Biol. 119, 493-501). Islet cultures are washed with PBS, fixed in 4% paraformaldehyde (30 min, room temperature) followed by permeabilization with 0.5% Triton X-100 (4 min, room temperature), followed by the TUNEL assay, performed according to the manufacturer's instructions (In Situ Cell Death Detection Kit, AP; Boehringer Mannheim, Germany). The preparations are then rinsed with Tris-buffered saline and is incubated (10 min, room temperature) with 5-bromo-4-chloro-indolyl phosphate/nitro blue tetrazolium liquid substrate system (Sigma). For staining of the activated caspase 3, after fixation and permeabilization, islets are incubated for 2 h at 37° C. with a rabbit anti-cleaved caspase-3 antibody (1:50 dilution, D 175; Cell Signaling, Beverly, Mass.), followed by incubation (30 min, 37° C.) with a Cy3-conjugated donkey anti-rabbit antibody (1:100 dilution; Jackson ImmunoResearch Laboratories, West Grove, Pa.). Thereafter, islets are incubated with a guinea pig anti-insulin antibody as described above, followed by detection using the streptavidin-biotin-peroxidase complex (Zymed) or by a 30-min incubation with a 1:20 dilution of fluoresceinconjugated rabbit anti-guinea pig antibody (Dako). The TUNEL assay detects DNA fragmentation associated with both apoptotic and necrotic cell death; therefore, islets are also treated with a fluorescent annexin V probe (Annexin-V-FLUOS staining kit, Boehringer Mannheim) according to the manufacturer's instructions. Double staining of cells with propidium iodide and annexin V enables the differentiation of apoptotic from necrotic cells.

### Example 8

### Anti-Inflammatory Applications

Zucker diabetic fatty rats are sacrificed and pancreatic islets are harvested as according to Shimabukuro et al. In culture, these islets are treated with an effective amount of Tumor Necrosis Factor alpha. De novo synthesis of ceramides is evaluated by incorporation of tritiated serine, as described in Example 8. Treatment with an effective concentration of compounds presented herein results in a significantly decreased concentration of ceramide in contrast to the control group. This demonstrates the efficacy of the compounds and specific inhibition activity against SPT in general, in anti-inflammatory applications.

### Example 9

### Serine Palmitoyltransferase Activity

Assay A.

The assay is carried out by a minor modification of the method reported by Merrill et al., Anal. Biochem., 171: 373-381 (1988).

Frozen rat or other mammalian livers are homogenized in a standard HEPES buffer system containing DTT (5 mM), sucrose (0.25 M) and EDTA at pH 7.4. The homogenate is spun at 30 kg for 0.5 hr. and the supernatant is removed. The assay is performed using the supernatant (sufficient for 50-150 μg protein) above but with the addition of 50 μM pyridoxal, 200 μM palmitoyl-CoA, and 1 mM ³H-L-serine in a buffer similar to the homogenization buffer, but at pH 8.3. The radiolabeled product, 3-ketosphinganine, is extracted in CHCl₃/CH₃OH and the radioactivity is counted in a liquid scintillation counter.

Inhibition of serine palmitoyl transferase is evaluated by incorporation of tritium label into the lipid product. Further demonstration of the activity of compounds in a CTLL-2 cell line can be performed using the assay described in Nakamura, S. et al., J. Biol. Chem., 271: 1255-7 (1996).

Assay B.

An alternative assay for evaluating inhibition of SPT, the enzyme present in commonly cultured cells, is performed with CHO cells or a human cell line. Cells are washed three times with ice-cold phosphate-buffered saline (PBS). A total of 0.5 mL of lysis buffer [50 mM Hepes (pH 8.0) containing 5 mM ethylenediaminetetraacetic acid (EDTA) and 5 mM dithiothreitol (DTT)] is added to each dish. The cells are scraped using a rubber policeman, and are then transferred to a test tube on ice. The cell suspension is sonicated three times for 5 s at 1-2 min intervals on ice. Protein concentrations in cell homogenates are measured using a Bradford protein assay kit (Bio-Rad). To measure the SPT activity, 0.1 mL of cell homogenates are added to 0.1 mL of reaction buffer [20 mM Hepes (pH 8.0) containing 5 mM EDTA, 10 mM DTT, 50 μM pyridoxal-5′-phosphate, 0.4 mM palmitoyl CoA, 2 mM L-serine, 10 μCi of [³H]serine, and test compound or standard inhibitor (myriocin). After incubation at 37° C. for 20 min with shaking, the reaction is terminated with 0.5 mL of 0.5 N NH₄OH containing 10 mM L-serine. The lipid products are extracted using the solvent system: 3 mL of chloroform/methanol (1:2), 25 μg of sphingosine (1 mg/mL in ethanol) as a carrier, 2 mL of chloroform, and 3.8 mL of 0.5 N NH₄OH. After vigorous mixing, the phases are separated by centrifugation at 2500 rpm for 5 min. The aqueous layer is removed by aspiration, and the lower chloroform layer is washed 3 times with 4.5 mL of water. The chloroform layer is transferred to a scintillation vial, and the solvent is evaporated under N₂ gas. The radioactivity is measured with a LS6000TA liquid scintillation counter (Beckman). Nonspecific conversion of [³H] serine to chloroform-soluble species is determined by performing the assay in the absence of palmitoyl CoA. The count of the background is about one-sixth of the count of 100% activity.

Assay C.

An alternative assay using a non-chlorinated solvent modification of the Blye and Dyer lipid extraction method reported in Smedes (Smedes, F. (1999) Analyst 124, 1711-18) is employed to evaluate exemplary compounds. In this approach, the cells are washed three times with ice-cold phosphate-buffered saline and 0.5 mL of lysis buffer is added to each dish. The cells are scraped using a rubber policeman and transfer to a test tube on ice. The cell suspension is sonicated three times for 5 s at 1-2 min intervals on ice. A 0.1 mL sample of cell homogenates are added to 0.1 mL of reaction buffer in a test tube containing the appropriate concentration of test substance and 10 μCi of [³H] serine. The reaction mixture is incubated at 37° C. for 20 min with shaking, and the reaction is terminated with 0.5 mL of 0.05N NH₄OH stop solution containing 10 mM unlabeled L-serine. Total lipids are extracted by transferring the contents of the test tube into a 15 ml centrifuge tube containing: 4.5 mL of isopropanol/cyclohexane (4:5) containing 25 pg of sphingosine (1 mg/mL in ethanol and diluted into the isopropanol/cyclohexane mixture) as a carrier. The contents are mixed vigorously and 4 mL of 0.5 N NH₄OH is added. The phases are separated by centrifugation at 2500 rpm for 5 min. An accurately measured portion of the organic layer (4.0 ml) is added to a scintillation vial with 1 ml of water. Ultima Gold F (5 ml) is added, the vial is vortexed and allowed to settle into separate layers. The amount of [³H] serine radioactivity incorporated into lipids is quantified in a scintillation counter. Non-specific counts are determined by carrying out the assay with control samples containing no palmitoyl CoA. As shown in Table 2 below, the positive control, ISP-1 (i.e., myriocin) exhibited potent but non-selective inhibition of SPT. Exemplary compound 12 is evaluated in this assay and, as shown in Table 2, exhibited moderate activity at the doses indicated.

TABLE 2

| Test group | Counts | Std Error |
|---|---|---|
| no CoA (blank) | 305 | 5 |
| No Inhibitor, t = 0 | 244 | 7 |

### Example 10

### Protection of Islets by an SPT Inhibitor

Islet protection by an exemplary compound is evaluated in an assay according to Eitel, K, et al. Biochem. Biophys. Res. Commun. 299: 853-6 (2002), and results obtained in this assay are reported below in Table 4. Rat pancreatic islets are cultured with control medium (RPMI 1640 supplemented with 10% fetal bovine serum, antibiotics and made 8% in glucose) or in medium supplemented with 1 millimolar sodium palmitate (Fatty Acid Medium) during a period of 3 days. The culture medium is changed after 2 days to an identical composition culture medium with fresh inhibitor in the appropriate wells. Cells are stained with propidium iodide (PI), washed and propidium staining of cells (as a measure of cellular DNA content) is assessed by flow cytometry. The percentage of cells having less than the normal amount of PI staining is considered to be apoptotic cells (Eitel, K, et al. (2002)).

In this assay, treatment with exemplary compound 12 appears to fully protect cells from the fatty acid treatment in this assay and surprisingly imparts a benefit in comparison to treatment with the control medium.

### REFERENCES

Ayasolla K., et al. Free Radic. Biol. Med. 37(3):325-38 (2004)
Beattie G M, Leibowitz G, Lopez A D, Levine F, Hayek A. (2000). Protection from cell death in cultured human fetal pancreatic cells. Cell Transplant. 9, 431-8
Beattie G M, Crowe J H, Lopez A D, Cirulli V, Ricordi C, and Hayek A (1997). Trehalose: a cryoprotectant that enhances recovery and preserves function of human pancreatic islets after long-term storage. Diabetes. 46:519-23.
Benjamins J A, et al. (2003). Protection of mature oligodendrocytes by inhibitors of caspases and calpains Neurochem Res. 28:143-52.
Bennett J W and Klich M. (2003). Clin Microbiol Rev. 16, 497-516.
Brinkmann V. et al., J Biol Chem 277: 21453-7 (2002).
Burke, S. E., Kuntz, R. E. and Schwartz, L. B. (2006) Zotarolimus (ABT-578) eluting stents. Adv. Drug Deliv. Rev. 58:437-46.
Burt, H. M. and Hunter, W. L. (2006) Drug-eluting stents: a multidisciplinary success story. Adv. Drug Deliv. Rev. 58: 350-7.
Byun, H.-S., Lu, X. and Bittman, R. (2006) Stereoselective Total synthesis of erine Palmitoyl-CoA Transferase Inhibitors. Synthesis: 2447-74.
Castellanos, E, Reyes-Rangel, G, Juaristi, E. Helv. Chim. Acta 87: 1016
Cativiela, C., and Diaz-de Villegas, M. D. Tetrahedron: Asymmetry 9: 3517-3599 (1998).
Chen, J K, et al. (1999). The identification of myriocin-binding proteins. Chem. Biol. 6, 221-35.
Chen, X., and Wang, W. (2003) Ann. Rep. Med. Chem. 38: 333
Chiba, K., Matsuyuki, H., Maeda, M., Sugahara, K. (2006) Role of Sphingosine 1-Phosphate Receptor Type 1 in Lymphocyte Egress from Secondary Lymphoid Tissues and Thymus. Cell. Molec. Immunol. 3: 11-19.
Clemens J. J. et al., (2004). Synthesis of benzimidazole based analogues of sphingosine-1-phosphate: discovery of potent, subtype-selective S1P4 receptor agonists. Bioorg. Med. Chem. Lett., 14: 4903-6.
Coroneos, E; Wang, Y; Panuska, J R; Templeton, D J; Kester, M. (1996). Biochem J, 31, 13-7.
Cutler R G, et al. (2004). Involvement of oxidative stress-induced abnormalities in ceramide and cholesterol metabolism in brain aging and Alzheimer's disease. Proc Natl. Acad. Sci. 101, 2070-5.
Ding, W X and Yin, X M, J. Cell. Mol. Med. 8, 445-54 (2004)
Durand, P. et al., (2000). Synthesis, 505, 6
Eitel, K., et al., (2002). Different role of saturated and unsaturated fatty acids in beta-cell apoptosis. Biochem Biophys Res Commun. 299, 853-6.
Esmon, C T. Crosstalk between inflammation and thrombosis. (2004) Maturitas. 47, 305-14
Frost R A and Lang C H. (2005). Curr Opin Clin Nutrit Metab Care, 255-263.
Fujita, T, et al. (1996). Potent Immunosuppressants, 2-alkyl-2-aminopropane-1,3-diols. J. Med. Chem. 39, 4451-59.
Gaspardone, A, et al. (2002) Effect of atorvastatin (80 mg) initiated at the time of coronary artery stent implantation on C-reactive protein and six-month clinical events. Am. J. Cardiol. 90:786-9.
Gaspardone A and Versaci, F. (2005) Coronary stenting and inflammation. Am. J. Cardiol. 96(12A): 65L-70L.

Gavrieli Y, et al. (1992). Identification of programmed cell death in situ via specific labeling of nuclear DNA fragmentation. J. Cell Biol. 119, 493-501.
Greene T and Wuts PGM. (1999) “Protective Groups in Organic Synthesis”, Edition 3, Wiley, New York.
Groth U, and Schöllkopf, U. Asymmetric Syntheses via Heterocyclic Intermediates XIX. Synthesis 1983: 673-5.
Grundy, S. M., et al. (2004) Definition of metabolic syndrome: Report of the National Heart, Lung, and Blood Institute/American Heart Association conference on scientific issues related to the definition. Circulation 109: 433-8.
Hale J J, et al. (2004). Synthesis, stereochemical determination and biochemical characterization of the enantiomeric phosphate esters of the novel immunosuppressive agent FTY720. Bio-org. Med. Chem. Lett., 12, 4803-7
Hale J J, et al., (2004). Potent S1P receptor agonists replicate the pharmacologic actions of the novel immune modulator FTY720. Bioorg. Med. Chem. Lett., 14, 3351-5.
Hanada K., et al., (2003). Biochem Biophys Acta, 1632: 16-30.
Hatakeyama, S, et al. J. Org. Chem. 62: 2775-9 (1997).
Hayes, C. J., et al. J. Org. Chem. 71: 2661-5 (2006).
Hinterding K, Albert, R and Cottens, S. First asymmetric synthesis of chiral analogues of the novel immunosuppressant FTY720. Tetrahedron Lett. 43: 8095-7 (2002)
Hojjati, M. R., Li, Z., Zhou, H., Tang, S., Huan, C., Ooi, E., Lu, S., Jiang, X. C. (2005.) Effect of myriocin on plasma sphingolipid metabolism and atherosclerosis in apoE-deficient mice. J. Biol. Chem. 280:10284-9.
Ilieva, A., Yuan, S., Wang, R. N., Agapitos, D., Hill, D. J., Rosenberg, L. (1999). Pancreatic islet cell survival following islet isolation: the role of cellular interactions in the pancreas. J Endocrinol. 161: 357-64.
Kajita, K., et al. Diabetes. Res. Clin. Pract. 66 Suppl 1, S79-83 (2004)
Kanzler, S., et al. Semin Cancer Biol. 10(3):173-84 (2000)
Kiuchi M., Adachi, K., Kohara, K. et al. (2000). Synthesis and Immunosuppressive Activity of 2-Substituted 2-Aminopropane-1,3-diols and 2-Aminoethanols. J. Med. Chem., 43, 2946-61.
Kobayashi, S., et al. (1998a). Catalytic Asymmetric Syntheses of Antifungal Sphingofungins and Their Biological Activity as Potent Inhibitors of Serine Palmitoyltransferase (SPT). J. Am. Chem. Soc. 120, 908-19.
Kobayashi, S, and Furuta, T. use of Heterocycles as Chiral Ligands and Auxiliaries in Asymmetric Syntheses of Sphingosine, Sphingofungins B and F. Tetrahedron 54: 10275-94 (1998b).
Kolb, H. C., et al. (1994). Chem. Rev. 94: 2483-2547.
Lane, J. W. and Halcomb, R. L. Org. Lett. 5: 4017-20 (2003).
Larock, R. C. (1999). “Comprehensive Organic Transformations” 2^{nd} Edition, Wiley, New York.
Lee, K. Y, et al. Tet. Lett. 43: 9361-9363 (2002).
Liao, J., Tao, J., Lin, G., Liu, D. Chemistry and biology of sphingolipids. Tetrahedron 61: 4715-33 (2005).
Lima, L. M. and Barreiro, E. J. (2005). Bioisosterism: A Useful Strategy for Molecular Modification and Drug Design. Curr. Med. Chem. 12: 23-49.
Maedler, K., et al. (2003). Monosaturated Fatty Acids Prevent the Deleterious Effects of Palmitate and High Glucose on Human Pancreatic β-Cell Turnover and Function. Diabetes; 52:726-33.
Mandala, S. et al., Science, 296: 346-9 (2002).
Matsumoto S, Goel S, Qualley S, Strong D M, Reems J A (2003). A comparative evaluation of culture conditions for short-term maintenance (<24 hr) of human islets isolated using the Edmonton protocol. Cell Tissue Bank. 4(2/4):85-93.
McTiernan, C F, et al. Curr Cardiol Rep. 2(3), 189-97 (2000)
Merrill et al., Anal. Biochem., 171: 373-381 (1988).
Meyer, S. G. and H. de Groot, Cycloserine and threo-dihydrosphingosine inhibit TNF-alpha-induced cytotoxicity: evidence for the importance of de novo ceramide synthesis in TNF-alpha signaling. Biochim Biophys Acta, 2003. 1643: p. 1-4.
Najera, T., et al. Eur. J. Org. Chem. 2000: 2809-20.
Nakamura, S. et al., (1996). J. Biol. Chem., 271: 1255-7.
Oishi, T., et al. (2001). Stereoselective total synthesis of (+)-myriocin from D-mannose. Chemical Commun. 1932-3.
Oberholzer J, et al. (2000). Human islet transplantation: lessons from 13 autologous and 13 allogeneic transplantations. Transplantation 69, 1115-1123.
Ohfune, Y. and Shinada, T. Eur. J. Org. Chem. 2005: 5127-43.
Ohgawara H, Mochizuki N, Karibe S, Omori Y. Survival and B-cell function of neonatal pig pancreatic islet-like cell clusters in an extracellular matrix. Pancreas. 6: 625-30 (1991).
Otonkoski T, Ustinov J, Rasilainen S, Kallio E, Korsgren O, Hayry P. Differentiation and maturation of porcine fetal islet cells in vitro and after transplantation. Transplantation. 68(11):1674-83 (1999).
Paraskevas S, Maysinger D, Wang R, Duguid T P, Rosenberg L (2000). Cell loss in isolated human islets occurs by apoptosis. Pancreas 2000, 20, 270-6.
Park, T S, Panek, R. L., Mueller, S. B., Hanselman, J. C., Rosebury, W. S., Robertson, A. W., Kindt, E. K., Homan, R., Karanthanasis, S. K., Rekhter, M. D. (2004) Inhibition of sphingomyelin synthesis reduces atherogenesis in apolipoprotein E-knockout mice. Circulation; 110:3465-71.
Park, T S, Panek, R. L., Rekhter, M. D., Mueller, S. B., Rosebury, W. S., Robertson, A. W, Hanselman, J. C. (2006). Modulation of lipoprotein metabolism by inhibition of sphingomyelin synthesis in ApoE knockout mice. Atherosclerosis, epub ahead of print.
Patani, G. A., and LaVoie, E. J. (1996) Chem Rev 96: 3147
Petasis, N. A. and Zavialov, I. A. (1997). A New and Practical Synthesis of α-Amino Acids from Alkenyl Boronic Acids. J. Am. Chem. Soc. 119: 445-6.
Petasis, N. A. and Zavialov, I. A. (1998). Highly Stereocontrolled One-Step Synthesis of anti-β-Amino Alcohols from Organoboronic Acids, Amines, and α-Hydroxy Aldehydes.
Petasis, N A (2005). “Multicomponent Reactions with Organoboron Compounds” In Multicomponent Reactions, pp 199-223, J. Zhu and H Bienayme, Eds., Wiley-VCH Verlag, Weinheim, Germany.
Petrache I., et al., Ceramide upregulation causes pulmonary cell apoptosis and emphysema-like disease in mice. Nat. Med. 11: 491-8 (2005).
Pileggi A, et al. (2004). Protecting pancreatic beta-cells. IUBMB Life 56, 387-94.
Pipik, B, et al. (2004). A Preferred Synthesis of 1,2,4-Oxadiazole. Synth. Commun. 34, 1863-70.
Prakash, G. K. S., Mandal, M, Schweizer, S, Petasis, N. A., and Olah, G. A. (2002). Stereoselective synthesis of anti-α-(Difluoromethyl)-β-amino Alcohols by Boronic Acid Based Three-Component Condensation. Stereoselective Preparation of (2S,3R)-Difluorothreonine. J. Org. Chem. 67: 3718-23.
Radke, P. W., et al. (2004) Dexamethasone and restenosis after coronary stent implantation. Curr. Pharm. Des. 10: 3449-55.
Rall L C and Roubenoff R Rheumatol 2004: 43, 1219-23.
Rosen H and Liao, J. (2003). Curr Opin Chem Biol 7: 461-8.
Rosenberg L, Wang R, Paraskevas S, Maysinger D. (1999). Structural and functional changes resulting from islet isolation lead to islet cell death. Surgery. 126: 393-8.
Rother K I, Harlan D M; J Clin Invest. 2004; 114: 877-83.
Ryan E A, Lakey J R, Rajotte R V, Korbutt G S, Kin T, Imes S, Rabinovitch A, Elliott J F, Bigam D, Kneteman N M, Warnock G L, Larsen I, and Shapiro A M (2001). Clinical outcomes and insulin secretion after islet transplantation with the Edmonton protocol. Diabetes 50:710-9.
Sano S., et al. Asymmetric Total Synthesis of ISP-1 (Myriocin, Thermozymocidin), a Potent Immunosuppressive Principle in the Isaria sinclairii Metabolite. Tetrahedron Lett 36: 2097-2100 (1995).
Sauerwald T M, et al. (2003). Study of caspase inhibitors for limiting death in mammalian cell culture. Biotechnol Bioeng. 81:329-40.
Sawada, M, et al. (2004). Cell Death Differ. 11, 997-1008.
Schimitz-Peiffer C. et al., (1999). J. Biol. Chem., 274: 24202-10.
Schöllkopf, U. Pure Appl Chem. 55: 1799 (1983).
Schöllkopf, U., et al. Asymmetric Syntheses via Heterocyclic Intermediates, X L. Liebigs Ann Chem. 1988: 781-6.
Seebach, D. et al. (1987). Stereoselektive Alkylierung an C(α) von Serin, Glycerinsaüre, Threonin und Weinsaüre über heterocycliische Enolate mit exocyclischer Doppelbindung. Helv. Chim. Acta. 70. 1194-1216.
Seidel G. et al. (2004). Iron-Catalyzed Cross-Coupling Reactions. A Scalable Synthesis of the Immunosuppressive Agent FTY720. J. Org. Chem., 69, 3950-52.
Shimabukuro et al., (1998). J. Biol. Chem., 273: 32487-90.
Shimabukuro M., et al., (1998). Proc. Natl. Acad. Sci. USA, 95: 2498-2502.
Shinoda J, et al. (1999). Cell Signal. 11: 435-41.
Smedes, F (1999). Determination of total lipid using non-chlorinated solvents. Analyst 124: 1711-18.
Smith S. C., et al. (2001) ACC/AHA Guidelines for Percutaneous Coronary Intervention (Revision of the 1993 PTCA Guidelines—Executive Summary. Circulation 103: 3019-3041.
Southwood, T. J., Curry, M. C., Hutton, C. A. Factors affecting the efficiency and stereoselectivity of α-amino acid synthesis by the Petasis Reaction. Tetrahedron 62: 236-42 (2006).
Spector, A A, et al. Analysis of long-chain free fatty acid binding to bovine serum albumin by determination of stepwise equilibrium constants. Biochemistry 10, 3226-32 (1971).
Sugiyama, S., Arai, S., Kiriyama, M., Ishii, K. Chem. Pharm. Bull. 53:100-2 (2005).
Tisdale M J. Langenbecks Arch Surg. 389: 299-305 (2004).
Takai, T., Nitta, K., Utimoto, K. et al. J Am Chem Soc 108: 7408 (1986).
Trost, B. M. and Lee, C. gem-Diacetates as Carbonyl surrogates for Asymmetric Synthesis. Total Synthesis of Sphingofungin E and F. J. Am. Chem. Soc. 123: 12191-201 (2001).
Trost, B. M. J. Org. Chem. 69: 5813-37 (2004).
Unger R. H. (2002). Lipotoxic diseases. Annu Rev Med 53: 319-36.
Viso, A., Fernandes de la Pradilla, R., Garcia, A. and Flores, A. α,β-Diamino acids: Biological Significance and Synthetic Approaches. Chem. Rev. 105: 3167-96 (2005).
Wang, Y.-F., et al. (1988). Lipase-catalyzed irreversible transesterifications using enol esters as acylating reagents: preparative enantio- and regioselective synthesis of alcohols, glycerol derivatives, sugars, and organometallics. J. Am. Chem. Soc. 110, 7200-5;
Wencker D. et al. (2003). J. Clin. Invest., 111:1497-1504.
Wittaker, D. R. and Fillinger, M. F. (2006). The engineering of endovascular stent technology: a review. Vasc. Endovascular Surg. 40: 85-94.
Yang, B., et al. Inhibitors directed towards caspase-1 and -3 are less effective than pan caspase inhibition in preventing renal proximal tubular cell apoptosis. Nephron Exp Nephrol. 96, e39-51 (2004).
Yang, C. and Burt, H. M. (2006) Drug-eluting stents: factors governing local pharmacokinetics. Adv. Drug Deliv. Rev. 58: 402-11.
Yang, B, El Nahas, A. M., Fisher, M., Wagner, B., Huang, L., Storie, I., Barnett, D., Barratt, J., Smith, A. C., Johnson, T. S. (2004). Inhibitors directed towards caspase-1 and -3 are less effective than pan caspase inhibition in preventing renal proximal tubular cell apoptosis. Nephron Exp Nephrol. 96(2):e39-51 (2004).
Zimmet P, et al. (2001). Nature, 414, 783-7.

What is claimed is:

1. A compound, and pharmaceutically acceptable salts thereof, corresponding to Formula (I): 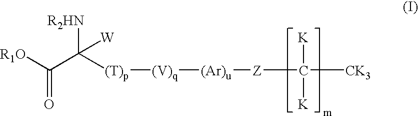
wherein:
R₁ is H, or optionally substituted lower alkyl, aryl, aralkyl, or alkyloxyalkyl;
R₂ is H, protecting group, or —C(═O)—CHRₐ—NHR_{b};
Rₐ is selected from the group consisting of alkyl, aralkyl, aryl, and optionally substituted alkyl with hydroxyl, halo, alkenyl, alkynl, ether, thiol, methylthio, borate, boronate, phospho, phosphono, phosphine, heterocyclic, enone, imine, aldehyde, ester, thioacid, hydroxylamine, amino group, and combinations thereof;
R_{b} is H or amino protecting group;
W is R₅ or selected from the group consisting of —CH₃, —CH₂CH₃, —CH₂X, —CHX₂, —CX₃, —CH₂CH₂X,
—CH₂CHX₂, —CH₂CX₃, —CH₂SO₂NH—R₃, —CH₂CH₂SO₂NH—R₃, —CH₂NHSO₂—R₃, —CH₂CH₂NHSO₂NH—R₃,
—CH₂SO₂—R₃, —CH₂CH₂SO₂—R₃, —C(O)OH, —CH₂C(O)OH, —CH₂C(O)NH—R₃, —CH₂CH₂C(O)NH—R₃, —CH₂NHC(O)—R₃,
—CH₂CH₂NHC(O)—R₃, —CH₂NHC(O)NH—R₃, —CH₂CH₂NHC(O)NH—R₃, —CH₂O—R₄, —CH₂CH₂O—R₄,
—CH₂NO₂, —CH₂CH₂NO₂, —CH₂CN, —CH₂CH₂CN, —CH₂OCX₃, —CH₂CH₂OCX₃, and —CH₂C(O)CX₃;
each X is independently a halogen;
R₃ is H, or optionally substituted lower alkyl, aryl, aralkyl, or alkyloxyalkyl;
R₄ is optionally substituted lower alkyl, aryl, aralkyl, or alkyloxyalkyl;
R₅ is optionally substituted lower alkyl, aryl, aralkyl, or alkyloxyalkyl, with the proviso that R₅ is not
—CH₂OR₂;
each V and Z is independently (CR_{c}R_{d})ₖ, CR_{c}═CR_{d}, O, NRₑ, S, Ar, CR_{c}R_{d}Ar, OAr, NR₄Ar,
SAr, or ArAr;
each R_{c}, and R_{d} is independently H, lower alkyl, OH, or O-lower alkyl;
or R_{c}, and R_{d} together form a ═O, ═N—OH, ═N—O-lower alkyl, or ═N—O—CH₂CH₂—O—CH₃;
Rₑ is H, lower alkyl, or —CH₂CH₂—O—CH₃;
k is 1 to 7;
q is 1 to 13;
each K is independently —H, —OH, —X, or —CH₃;
each T is independently (CR_{f}R_{g});
each R_{f} is independently H, lower alkyl, or O-lower alkyl;
each R₉ is independently OH, or O-lower alkyl;
or R_{f} and R_{g}, together form a ═O, ═N—OH, ═N—O-lower alkyl, or ═N—O—CH₂CH₂—O—CH₃;
p is 1 to 5;
each Ar is an optionally substituted aryl or heteroaryl;
u is 0, 1, or 2; and
m is 4 to 12.

2. The compound of claim 1, corresponding to Formula (II): 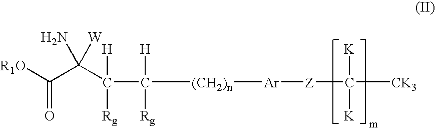
wherein n is 0 to 7.

3. The compound of claim 1, corresponding to Formula (III): 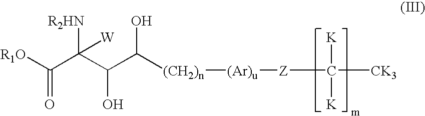
wherein n is 0 to 7.

4. The compound of claim 1, corresponding to Formula (IIIA): 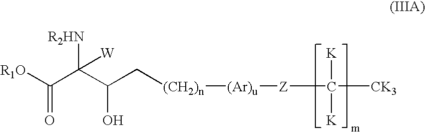
wherein n is 0 to 7.

5. The compound of claim 1, corresponding to Formula (IIIB): 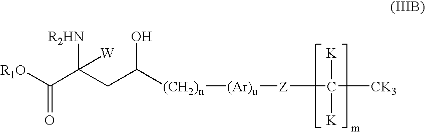
wherein n is 0 to 7.

6. The compound of claim 1, wherein each Ar is independently an optionally-substituted phenyl, pyridinyl, pyrimidyl, imidazolyl, benzimidazolyl, thiazolyl, oxazolyl, isoxazolyl, benzthiazolyl, or benzoxazolyl.

7. The compound of claim 6, wherein each Ar is independently phenyl, pyridinyl, or oxazolyl.

8. The compound of claim 1, wherein W is selected from the group consisting of —CH₂SO₂NH₂, —CH₂C(O)NH₂,
—CH₂SO₂NHCH₃, —CH₂CN, —CH₂CF₃, —CH₂OCH₃, —CH₂OCF₃, —CH₃, —CH₂F, —CF₂H, —CH₂CCl₃, —CH₂C(O)NHCH₃,
—CH₂SO₂CH₃, —CH₂NHC(O)H, —CH₂NHC(O)NH₂, and —CH₂C(O)CF₃.

9. The compound of claim 1, wherein each X is fluorine.

10. The compound of claim 1, wherein R₁ is C₁-C₃ alkyl.

11. The compound of claim 1, wherein R₁ is CH₃—O—CH₂—CH₂—, HO—CH₂—CH₂—, HO—CH₂—CH₂—O—CH₂—CH₂—, or CH₃—O—CH₂—CH₂—O—CH₂—CH₂—.

12. The compound of claim 1, wherein p is 2.

13. The compound of claim 1, wherein said compound modulates Serine Palmitoyltransferase (SPT) activity.

14. The compound of claim 13, wherein said compound inhibits Serine Palmitoyltransferase (SPT).

15. The compound of claim 13, wherein said compound does not cause strong immunosuppressive activity.

16. A composition comprising the compound of claim 1 and a pharmaceutically acceptable carrier.

17. A composition comprising the compound of claim 1 and a therapeutically effective amount of at least one active agent selected from the group consisting of insulin, insulin analogs, incretin, incretin analogs, glucagon-like peptide, glucagon-like peptide analogs, exendin, exendin analogs, PACAP and VIP analogs, sulfonylureas, biguanides, α-glucosidase inhibitors, Acetyl-CoA Carboxylase inhibitors, caspase inhibitors, and PPAR ligands.

18. A method of treating insulin resistance, said method comprising administering the compound of claim 1 to a patient in need thereof.

19. A method of treating pancreatic beta cell apoptosis, said method comprising administering the compound of claim 1 to a patient in need thereof.

20. A method of treating obesity, said method comprising administering the compound of claim 1 to a patient in need thereof.

21. A method of treating pro-thrombotic conditions, myocardial infarction, hypertension, dyslipidemia, or other manifestations of Syndrome X, said method comprising administering the compound of claim 1 to a patient in need thereof.

22. A method of treating congestive heart failure, said method comprising administering the compound of claim 1 to a patient in need thereof.

23. A method of treating an inflammatory disease, said method comprising administering the compound of claim 1 to a patient in need thereof, wherein said inflammatory disease is a disease of the cardiovascular system, atherosclerosis, or sepsis.

24. A method of preventing loss or death of human or xenobiotic islet cells in culture fluid, said method comprising adding a compound of claim 1 to the culture fluid.

25. A method for preserving liver tissue in culture fluid, said method comprising adding a compound of claim 1 to the culture fluid.

26. A method for treatment or prevention of type 1 diabetes, said method comprising administering the compound of claim 1 to a patient in need thereof.

27. A method for treatment or prevention of liver damage, said method comprising administering the compound of claim 1 to a patient in need thereof.

28. A method for treatment or prevention of cachexia, said method comprising administering the compound of claim 1 to a patient in need thereof.

29. A method for treatment or prevention of atherosclerosis, said method comprising administering the compound of claim 1 to a patient in need thereof.

30. A method for treating restenosis following percutaneous coronary intervention, comprising administering a therapeutically effective amount of at least one compound of claim 1 to a patient in need thereof.

31. A method for treating emphysema and chronic obstructive pulmonary disease, said method comprising administering a therapeutically effective amount of the compound of claim 1 to a patient in need thereof.

32. A device for percutaneous coronary intervention, comprising a controlled release formulation for administering a therapeutically effective amount of at least one compound of claim 1 to a patient in need thereof.

33. A method according to claim 18, further comprising co-administering a therapeutically effective amount of at least one active agent selected from the group consisting of insulin, insulin analogs, incretin, incretin analogs, glucagon-like peptide, glucagon-like peptide analogs, exendin, exendin analogs, PACAP and VIP analogs, sulfonylureas, biguanides, α-glucosidase inhibitors, Acetyl-CoA Carboxylase inhibitors, caspase inhibitors, unsaturated fatty acids, polyunsaturated fatty acids, HMG-CoA inhibitors, PPAR ligands, inhaled formulations containing bronchodilators, beta 2 adrenoceptor agonists, inhaled corticosteroids, anti-inflammatory steroids, leukotriene modifiers, leukotriene receptor antagonists, chemokine modifiers, chemokine receptor antagonists, cromolyn, nedocromil, xanthines, anticholinergic agents, immune modulating agents, other known anti-asthma medications, phosphodiesterase inhibitors, and other known anti-inflammatory medications.

34. A method according to claim 19, further comprising co-administering a therapeutically effective amount of at least one active agent selected from the group consisting of insulin, insulin analogs, incretin, incretin analogs, glucagon-like peptide, glucagon-like peptide analogs, exendin, exendin analogs, PACAP and VIP analogs, sulfonylureas, biguanides, α-glucosidase inhibitors, Acetyl-CoA Carboxylase inhibitors, caspase inhibitors, unsaturated fatty acids, polyunsaturated fatty acids, HMG-CoA inhibitors, PPAR ligands, inhaled formulations containing bronchodilators, beta 2 adrenoceptor agonists, inhaled corticosteroids, anti-inflammatory steroids, leukotriene modifiers, leukotriene receptor antagonists, chemokine modifiers, chemokine receptor antagonists, cromolyn, nedocromil, xanthines, anticholinergic agents, immune modulating agents, other known anti-asthma medications, phosphodiesterase inhibitors, and other known anti-inflammatory medications.

35. A method according to claim 20, further comprising co-administering a therapeutically effective amount of at least one active agent selected from the group consisting of insulin, insulin analogs, incretin, incretin analogs, glucagon-like peptide, glucagon-like peptide analogs, exendin, exendin analogs, PACAP and VIP analogs, sulfonylureas, biguanides, α-glucosidase inhibitors, Acetyl-CoA Carboxylase inhibitors, caspase inhibitors, unsaturated fatty acids, polyunsaturated fatty acids, HMG-CoA inhibitors, PPAR ligands, inhaled formulations containing bronchodilators, beta 2 adrenoceptor agonists, inhaled corticosteroids, anti-inflammatory steroids, leukotriene modifiers, leukotriene receptor antagonists, chemokine modifiers, chemokine receptor antagonists, cromolyn, nedocromil, xanthines, anticholinergic agents, immune modulating agents, other known anti-asthma medications, phosphodiesterase inhibitors, and other known anti-inflammatory medications.

36. A method according to claim 21, further comprising co-administering a therapeutically effective amount of at least one active agent selected from the group consisting of insulin, insulin analogs, incretin, incretin analogs, glucagon-like peptide, glucagon-like peptide analogs, exendin, exendin analogs, PACAP and VIP analogs, sulfonylureas, biguanides, α-glucosidase inhibitors, Acetyl-CoA Carboxylase inhibitors, caspase inhibitors, unsaturated fatty acids, polyunsaturated fatty acids, HMG-CoA inhibitors, PPAR ligands, inhaled formulations containing bronchodilators, beta 2 adrenoceptor agonists, inhaled corticosteroids, anti-inflammatory steroids, leukotriene modifiers, leukotriene receptor antagonists, chemokine modifiers, chemokine receptor antagonists, cromolyn, nedocromil, xanthines, anticholinergic agents, immune modulating agents, other known anti-asthma medications, phosphodiesterase inhibitors, and other known anti-inflammatory medications.

37. A method according to claim 22, further comprising co-administering a therapeutically effective amount of at least one active agent selected from the group consisting of insulin, insulin analogs, incretin, incretin analogs, glucagon-like peptide, glucagon-like peptide analogs, exendin, exendin analogs, PACAP and VIP analogs, sulfonylureas, biguanides, α-glucosidase inhibitors, Acetyl-CoA Carboxylase inhibitors, caspase inhibitors, unsaturated fatty acids, polyunsaturated fatty acids, HMG-CoA inhibitors, PPAR ligands, inhaled formulations containing bronchodilators, beta 2 adrenoceptor agonists, inhaled corticosteroids, anti-inflammatory steroids, leukotriene modifiers, leukotriene receptor antagonists, chemokine modifiers, chemokine receptor antagonists, cromolyn, nedocromil, xanthines, anticholinergic agents, immune modulating agents, other known anti-asthma medications, phosphodiesterase inhibitors, and other known anti-inflammatory medications.

38. A method according to claim 23, further comprising co-administering a therapeutically effective amount of at least one active agent selected from the group consisting of insulin, insulin analogs, incretin, incretin analogs, glucagon-like peptide, glucagon-like peptide analogs, exendin, exendin analogs, PACAP and VIP analogs, sulfonylureas, biguanides, α-glucosidase inhibitors, Acetyl-CoA Carboxylase inhibitors, caspase inhibitors, unsaturated fatty acids, polyunsaturated fatty acids, HMG-CoA inhibitors, PPAR ligands, inhaled formulations containing bronchodilators, beta 2 adrenoceptor agonists, inhaled corticosteroids, anti-inflammatory steroids, leukotriene modifiers, leukotriene receptor antagonists, chemokine modifiers, chemokine receptor antagonists, cromolyn, nedocromil, xanthines, anticholinergic agents, immune modulating agents, other known anti-asthma medications, phosphodiesterase inhibitors, and other known anti-inflammatory medications.

39. A method according to claim 24, further comprising co-administering a therapeutically effective amount of at least one active agent selected from the group consisting of insulin, insulin analogs, incretin, incretin analogs, glucagon-like peptide, glucagon-like peptide analogs, exendin, exendin analogs, PACAP and VIP analogs, sulfonylureas, biguanides, α-glucosidase inhibitors, Acetyl-CoA Carboxylase inhibitors, caspase inhibitors, unsaturated fatty acids, polyunsaturated fatty acids, HMG-CoA inhibitors, PPAR ligands, inhaled formulations containing bronchodilators, beta 2 adrenoceptor agonists, inhaled corticosteroids, anti-inflammatory steroids, leukotriene modifiers, leukotriene receptor antagonists, chemokine modifiers, chemokine receptor antagonists, cromolyn, nedocromil, xanthines, anticholinergic agents, immune modulating agents, other known anti-asthma medications, phosphodiesterase inhibitors, and other known anti-inflammatory medications.

40. A method according to claim 25, further comprising co-administering a therapeutically effective amount of at least one active agent selected from the group consisting of insulin, insulin analogs, incretin, incretin analogs, glucagon-like peptide, glucagon-like peptide analogs, exendin, exendin analogs, PACAP and VIP analogs, sulfonylureas, biguanides, α-glucosidase inhibitors, Acetyl-CoA Carboxylase inhibitors, caspase inhibitors, unsaturated fatty acids, polyunsaturated fatty acids, HMG-CoA inhibitors, PPAR ligands, inhaled formulations containing bronchodilators, beta 2 adrenoceptor agonists, inhaled corticosteroids, anti-inflammatory steroids, leukotriene modifiers, leukotriene receptor antagonists, chemokine modifiers, chemokine receptor antagonists, cromolyn, nedocromil, xanthines, anticholinergic agents, immune modulating agents, other known anti-asthma medications, phosphodiesterase inhibitors, and other known anti-inflammatory medications.

41. A method according to claim 26, further comprising co-administering a therapeutically effective amount of at least one active agent selected from the group consisting of insulin, insulin analogs, incretin, incretin analogs, glucagon-like peptide, glucagon-like peptide analogs, exendin, exendin analogs, PACAP and VIP analogs, sulfonylureas, biguanides, α-glucosidase inhibitors, Acetyl-CoA Carboxylase inhibitors, caspase inhibitors, unsaturated fatty acids, polyunsaturated fatty acids, HMG-CoA inhibitors, PPAR ligands, inhaled formulations containing bronchodilators, beta 2 adrenoceptor agonists, inhaled corticosteroids, anti-inflammatory steroids, leukotriene modifiers, leukotriene receptor antagonists, chemokine modifiers, chemokine receptor antagonists, cromolyn, nedocromil, xanthines, anticholinergic agents, immune modulating agents, other known anti-asthma medications, phosphodiesterase inhibitors, and other known anti-inflammatory medications.

42. A method according to claim 27, further comprising co-administering a therapeutically effective amount of at least one active agent selected from the group consisting of insulin, insulin analogs, incretin, incretin analogs, glucagon-like peptide, glucagon-like peptide analogs, exendin, exendin analogs, PACAP and VIP analogs, sulfonylureas, biguanides, α-glucosidase inhibitors, Acetyl-CoA Carboxylase inhibitors, caspase inhibitors, unsaturated fatty acids, polyunsaturated fatty acids, HMG-CoA inhibitors, PPAR ligands, inhaled formulations containing bronchodilators, beta 2 adrenoceptor agonists, inhaled corticosteroids, anti-inflammatory steroids, leukotriene modifiers, leukotriene receptor antagonists, chemokine modifiers, chemokine receptor antagonists, cromolyn, nedocromil, xanthines, anticholinergic agents, immune modulating agents, other known anti-asthma medications, phosphodiesterase inhibitors, and other known anti-inflammatory medications.

43. A method according to claim 28, further comprising co-administering a therapeutically effective amount of at least one active agent selected from the group consisting of insulin, insulin analogs, incretin, incretin analogs, glucagon-like peptide, glucagon-like peptide analogs, exendin, exendin analogs, PACAP and VIP analogs, sulfonylureas, biguanides, α-glucosidase inhibitors, Acetyl-CoA Carboxylase inhibitors, caspase inhibitors, unsaturated fatty acids, polyunsaturated fatty acids, HMG-CoA inhibitors, PPAR ligands, inhaled formulations containing bronchodilators, beta 2 adrenoceptor agonists, inhaled corticosteroids, anti-inflammatory steroids, leukotriene modifiers, leukotriene receptor antagonists, chemokine modifiers, chemokine receptor antagonists, cromolyn, nedocromil, xanthines, anticholinergic agents, immune modulating agents, other known anti-asthma medications, phosphodiesterase inhibitors, and other known anti-inflammatory medications.

44. A method according to claim 29, further comprising co-administering a therapeutically effective amount of at least one active agent selected from the group consisting of insulin, insulin analogs, incretin, incretin analogs, glucagon-like peptide, glucagon-like peptide analogs, exendin, exendin analogs, PACAP and VIP analogs, sulfonylureas, biguanides, α-glucosidase inhibitors, Acetyl-CoA Carboxylase inhibitors, caspase inhibitors, unsaturated fatty acids, polyunsaturated fatty acids, HMG-CoA inhibitors, PPAR ligands, inhaled formulations containing bronchodilators, beta 2 adrenoceptor agonists, inhaled corticosteroids, anti-inflammatory steroids, leukotriene modifiers, leukotriene receptor antagonists, chemokine modifiers, chemokine receptor antagonists, cromolyn, nedocromil, xanthines, anticholinergic agents, immune modulating agents, other known anti-asthma medications, phosphodiesterase inhibitors, and other known anti-inflammatory medications.

45. A method according to claim 30, further comprising co-administering a therapeutically effective amount of at least one active agent selected from the group consisting of insulin, insulin analogs, incretin, incretin analogs, glucagon-like peptide, glucagon-like peptide analogs, exendin, exendin analogs, PACAP and VIP analogs, sulfonylureas, biguanides, α-glucosidase inhibitors, Acetyl-CoA Carboxylase inhibitors, caspase inhibitors, unsaturated fatty acids, polyunsaturated fatty acids, HMG-CoA inhibitors, PPAR ligands, inhaled formulations containing bronchodilators, beta 2 adrenoceptor agonists, inhaled corticosteroids, anti-inflammatory steroids, leukotriene modifiers, leukotriene receptor antagonists, chemokine modifiers, chemokine receptor antagonists, cromolyn, nedocromil, xanthines, anticholinergic agents, immune modulating agents, other known anti-asthma medications, phosphodiesterase inhibitors, and other known anti-inflammatory medications.

46. A method according to claim 31, further comprising co-administering a therapeutically effective amount of at least one active agent selected from the group consisting of insulin, insulin analogs, incretin, incretin analogs, glucagon-like peptide, glucagon-like peptide analogs, exendin, exendin analogs, PACAP and VIP analogs, sulfonylureas, biguanides, α-glucosidase inhibitors, Acetyl-CoA Carboxylase inhibitors, caspase inhibitors, unsaturated fatty acids, polyunsaturated fatty acids, HMG-CoA inhibitors, PPAR ligands, inhaled formulations containing bronchodilators, beta 2 adrenoceptor agonists, inhaled corticosteroids, anti-inflammatory steroids, leukotriene modifiers, leukotriene receptor antagonists, chemokine modifiers, chemokine receptor antagonists, cromolyn, nedocromil, xanthines, anticholinergic agents, immune modulating agents, other known anti-asthma medications, phosphodiesterase inhibitors, and other known anti-inflammatory medications.
